(19) United States
(12) Patent Application Publication
(10) Pub. No.: US 2025/0331760 A1
(43) Pub. Date: Oct. 30, 2025

(54) Detection of Aortic Valve Stenosis From 12 Lead ECG Using Feed Forward Network

(71) Applicant: AccurKardia, Inc., New York, NY (US)

(72) Inventors: Sluso Anne Bose, Kerala (IN); Catherine Vinnarasi Antony, Tamil Nadu (IN); Komala T.A, Karnataka (IN); Rozina Moazzam Ali Shaikh, Maharashtra (IN)

(21) Appl. No.: 19/192,827

(22) Filed: Apr. 29, 2025

Related U.S. Application Data
(60) Provisional application No. 63/640,857, filed on Apr. 30, 2024.
(60) Provisional application No. 63/733,521, filed on Dec. 13, 2024.

(51) Int. Cl. A61B 5/36; G16H 50/20

(52) U.S. Cl. CPC A61B 5/36; G16H 50/20

(57) ABSTRACT

The present disclosure provides systems and methods for detection of aortic valve stenosis (AVS) from parameters derived from one or more electrocardiogram (ECG) leads. In particular, the present disclosure identified critical novel features that can be incorporated in systems and methods for the detection of aortic valve stenosis (AVS) from such parameters.

### FIELD

The present disclosure relates to systems and processes for detection of aortic valve stenosis (AVS). Aortic valve stenosis (AVS) is a cardiovascular condition characterized by the narrowing of the aortic valve opening, which obstructs the flow of blood from the left ventricle to the aorta, thereby impeding the efficient delivery of oxygenated blood to the body. This narrowing occurs due to the thickening and calcification of the valve leaflets, leading to reduced cardiac output and potentially life-threatening complications.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In some aspects, the disclosure provides a process for screening an ECG for a pattern that is predictive of aortic valve stenosis, the process comprising: screening a plurality of parameters from one or more electrocardiogram (ECG) leads for a pattern that is predictive of aortic valve stenosis, whereby the screening is performed by a computer program model trained to identify the predictive pattern from the plurality of parameters, whereby: at least a first parameter in the plurality of parameters is an age of a subject associated with the ECG; and at least a second parameter is selected from one or more of P wave amplitude, R wave amplitude, R wave duration, S wave amplitude, S wave duration, T wave amplitude, and QT interval, and outputting a value that is predictive of aortic valve stenosis based on the pattern that is predictive of aortic valve stenosis. A plurality of additional parameters can contribute to the pattern that is predictive of aortic valve stenosis, in some cases the at least one second parameter is the QT interval, the at least one additional parameter is a T amplitude from lead V4, the at least one additional parameter is a T amplitude from lead AVL, the at least one additional parameter is a R amplitude from lead II, the at least one additional parameter is a P wave amplitude of lead I, the at least one additional parameter is a P wave amplitude of lead II, the at least one additional parameter is a P wave amplitude of lead III, the at least one additional parameter is a P wave amplitude of lead V1, the at least one additional parameter is a P wave amplitude of lead V2, the at least one additional parameter is a P wave amplitude of lead V3, the at least one additional parameter is a P wave amplitude of lead V4, the at least one additional parameter is a P wave amplitude of lead V5, the at least one additional parameter is a P wave amplitude of lead V6, the at least one additional parameter is a P wave amplitude of lead aVF, the at least one additional parameter is a P wave amplitude of lead aVR, the at least one additional parameter is a P wave amplitude of lead aVL, the at least one additional parameter is a R wave amplitude of lead I, the at least one additional parameter is a R wave amplitude of lead III, the at least one additional parameter is a R wave amplitude of lead V1, the at least one additional parameter is a R wave amplitude of lead V2, the at least one additional parameter is a R wave amplitude of lead V3, the at least one additional parameter is a R wave amplitude of lead V4, the at least one additional parameter is a R wave amplitude of lead V5, the at least one additional parameter is a R wave amplitude of lead V6, the at least one additional parameter is a R wave amplitude of lead aVF, the at least one additional parameter is a R wave amplitude of lead aVR, the at least one additional parameter is a R wave amplitude of lead aVL, the at least one additional parameter is a R wave duration of lead aVL, the at least one additional parameter is a R wave duration of lead I, the at least one additional parameter is a R wave duration of lead II, the at least one additional parameter is a R wave duration of lead III, the at least one additional parameter is a R wave duration of lead V1, the at least one additional parameter is a R wave duration of lead V2, the at least one additional parameter is a R wave duration of lead V3, the at least one additional parameter is a R wave duration of lead V4, the at least one additional parameter is a R wave duration of lead V5, the at least one additional parameter is a R wave duration of lead V6, the at least one additional parameter is a R wave duration of lead aVF, the at least one additional parameter is a R wave duration of lead aVR, the at least one additional parameter is a R wave duration of lead aVL, the at least one additional parameter is a S wave amplitude of lead I, the at least one additional parameter is a S wave amplitude of lead II, the at least one additional parameter is a S wave amplitude of lead III, the at least one additional parameter is a S wave amplitude of lead V1, the at least one additional parameter is a S wave amplitude of lead V2, the at least one additional parameter is a S wave amplitude of lead V3, the at least one additional parameter is a S wave amplitude of lead V4, the at least one additional parameter is a S wave amplitude of lead V5, the at least one additional parameter is a S wave amplitude of lead V6, the at least one additional parameter is a S wave amplitude of lead aVF, the at least one additional parameter is a S wave amplitude of lead aVR, the at least one additional parameter is a S wave amplitude of lead aVL, the at least one additional parameter is a T wave amplitude of lead I, the at least one additional parameter is a T wave amplitude of lead II, the at least one additional parameter is a T wave amplitude of lead III, the at least one additional parameter is a T wave amplitude of lead V1, the at least one additional parameter is a T wave amplitude of lead V2, the at least one additional parameter is a T wave amplitude of lead V3, the at least one additional parameter is a T wave amplitude of lead V5, the at least one additional parameter is a T wave amplitude of lead V6, the at least one additional parameter is a T wave amplitude of lead aVF, the at least one additional parameter is a T wave amplitude of lead aVR. In some cases, the pattern that is predictive of aortic valve stenosis is a reduced QT interval as a function of the age of the subject associated with the ECG. In some cases, a negative predictive value of the pattern is greater than 90%. The aortic valve stenosis can have distinct etiologies, in some instances the aortic valve stenosis can be rheumatic disorder of both mitral and aortic valve, nonrheumatic AVS, nonrheumatic AVS with insufficiency, congenital stenosis of AVS, bicuspid aortic valve, congenital insufficiency of aortic valve, aortic insufficiency/stenosis, nonrheumatic aortic valve disorder, unspecified other nonrheumatic AVS, stenosis aortic moderate, stenosis aortic congenital, regurgitation aortic, rheumatic aortic stenosis with insufficiency, congenital subaortic stenosis, supravalvular aortic stenosis, rheumatic aortic stenosis with insufficiency, stenosis aortic rheumatic, aortic valve disease, aortic regurgitation, and rheumatic aortic stenosis. In some instances, the sensitivity and/or the specificity of the value that is predictive of aortic valve stenosis is greater than 70%. The computer program can be trained on a dataset that comprises at least 1,000 ECG's of subjects with at least one form of AVS and at least 1,000 control subjects who did not have a diagnosis of heart disease, or another suitable number. In some implementations, the process further comprises a step of treating a subject associated with the ECG for the aortic valve stenosis. In some configurations the computer program model is a feedforward neural network model, e.g., a computer program model trained on 62 parameters comprising: the age of a subject associated with the ECG, the P wave amplitude, the R wave amplitude, the R wave duration, the S wave amplitude, the S wave duration, the T wave amplitude, and the average QT. In some cases, a user directs the training of the model to adjust the learning rate dynamically based on the plateauing of a monitored metric. In some cases, a user directs the training of the model to prevent overfitting by monitoring validation metrics. In some cases, the ECG is from a subject, e.g., a human that is at least 18, at least 19, at least 20, at least 21, or at least 22 years or older. In some cases, the subject does not have a pacemaker. In some cases, the subject has not suffered a prior myocardial infarction, a left ventricular hypertrophy, or cardiac surgery. In some aspects, the output further comprises a risk score indicating the probability of developing moderate or severe aortic stenosis within a predefined time period following a negative echocardiogram. In some aspects, the output further comprises a risk score indicating the probability of developing heart failure within a predefined time period following a negative echocardiogram. In some aspects, the computer program model is further configured to analyze longitudinal ECG data from a subject and to classify the subject into a risk trajectory cluster, wherein the risk trajectory cluster is associated with a distinct prognosis for mortality or adverse cardiovascular outcomes. In some aspects, the computer program model is further configured to analyze periprocedural changes in the risk score before and after aortic valve intervention, and to output a prognostic indicator of 1-year mortality, risk of permanent pacemaker implantation, or length of hospital stay. In some aspects, a positive risk score in the absence of echocardiographic evidence of aortic stenosis is associated with a statistically significant increased risk of developing moderate or severe aortic stenosis or heart failure within five years. In some aspects, the output is used to trigger automated alerts or referrals for further diagnostic evaluation or early intervention in a hospital electronic health record system. In some aspects, the computer program model is trained and validated on datasets comprising at least 100,000 patients and is configured to maintain predictive accuracy across diverse demographic groups. In some aspects, the output further comprises a recommendation for timing of aortic valve intervention based on the subject's risk trajectory cluster and predicted clinical outcomes. In some aspects, the computer program model is further configured to provide a risk score for adverse outcomes following transcatheter aortic valve replacement, including mortality, need for permanent pacemaker, and length of hospital stay. In some aspects, the computer program model is further configured to provide a risk score for future onset of aortic stenosis in subjects with false-positive screening results, and wherein the risk score is used to guide longitudinal surveillance.

In some instances, the disclosure describes a system for screening an ECG for a pattern that is predictive of aortic valve stenosis, the system comprising: an input module (e.g., computer software module) for receiving a plurality of parameters from one or more electrocardiogram (ECG) leads; an analysis module (e.g., computer software module) comprising a computer program model trained to identify a pattern predictive of aortic valve stenosis from the plurality of parameters from at least two inputs: an age of a subject associated with the ECG; and a QT interval, and an output module (e.g., computer software module) for outputting a predictive of aortic valve stenosis based on the pattern predictive of aortic stenosis from the at least two inputs. In some instances, the analysis module identifies the pattern predictive of aortic valve stenosis from no more than two said inputs: the age of the subject associated with the ECG and the QT interval. In some instances, a negative predictive value of the pattern predictive of aortic valve stenosis is greater than 90%. In some instances, a sensitivity and or a specificity of the identification of the pattern that is predictive of aortic valve stenosis is greater than 70%. In some instances, the analysis module identifies a pattern predictive of aortic valve stenosis from at least three inputs selected from the group consisting of the age of the subject associated with the ECG, the QT interval, a T amplitude from lead V4, a T amplitude from lead AVL, and R amplitude from lead II. In some instances, the pattern that is predictive of aortic valve stenosis is a reduced QT interval as a function of the age of the subject associated with the ECG. In some instances, the pattern that is predictive of aortic valve stenosis is an increased T amplitude from lead V4 as a function of the age of the subject associated with the ECG. In some cases, the aortic valve stenosis is rheumatic disorder of both mitral and aortic valve, nonrheumatic AVS, nonrheumatic AVS with insufficiency, congenital stenosis of AVS, bicuspid aortic valve, congenital insufficiency of aortic valve, aortic insufficiency/stenosis, nonrheumatic aortic valve disorder, unspecified other nonrheumatic AVS, stenosis aortic moderate, stenosis aortic congenital, regurgitation aortic, rheumatic aortic stenosis with insufficiency, congenital subaortic stenosis, supravalvular aortic stenosis, rheumatic aortic stenosis with insufficiency, stenosis aortic rheumatic, aortic valve disease, aortic regurgitation, and rheumatic aortic stenosis. In some instances, the computer program is trained on a dataset that comprises at least 1,000 ECG's of subjects with at least one form of AVS and at least 1,000 control subjects who did not have a diagnosis of heart disease. In some instances, the computer program model is a feedforward neural network model. In some instances, the computer program is trained on 62 parameters, including ECG parameters such as P wave amplitude, R wave amplitude, R wave duration, S wave amplitude, S wave duration, T wave amplitude, and average QT interval. Feature selection is performed using a recursive feature elimination (RFE) algorithm with cross-validation to identify the most relevant features. Specifically, the RFE algorithm iteratively removes the least important features based on the weights assigned by a linear SVM classifier, and the cross-validation performance is used to determine the optimal subset of features. In some instances, a user directs the training of the model to adjusts the learning rate dynamically based on the plateauing of a monitored metric. In some instances, a user directs the training of the model to prevent overfitting by monitoring validation metrics. In some cases, the system screens the ECG patterns from a subject, e.g., a human, that is at least 18, at least 19, at least 20, at least 21, or at least 22 years or older. In some cases, the subject does not have a pacemaker. In some cases, the subject has not suffered a prior myocardial infarction, a left ventricular hypertrophy, or cardiac surgery. In some aspects, the output module is further configured to provide a risk score indicating the probability of developing moderate or severe aortic stenosis within a predefined time period following a negative echocardiogram. In some aspects, the output module is further configured to provide a risk score indicating the probability of developing heart failure within a predefined time period following a negative echocardiogram. In some aspects, the analysis module is further configured to analyze longitudinal ECG data from a subject and to classify the subject into a risk trajectory cluster, wherein the risk trajectory cluster is associated with a distinct prognosis for mortality or adverse cardiovascular outcomes. In some aspects, the analysis module is further configured to analyze periprocedural changes in the risk score before and after aortic valve intervention, and the output module is configured to provide a prognostic indicator of one-year mortality, risk of permanent pacemaker implantation, or length of hospital stay. In some aspects, the output module is configured such that a positive risk score in the absence of echocardiographic evidence of aortic stenosis is associated with a statistically significant increased risk of developing moderate or severe aortic stenosis or heart failure within five years. In some aspects, the output module is further configured to trigger automated alerts or referrals for further diagnostic evaluation or early intervention in a hospital electronic health record system based on the output. In some aspects, the computer program model is trained and validated on datasets comprising at least 100,000 patients and is configured to maintain predictive accuracy across diverse demographic groups. In some aspects, the output module is further configured to provide a recommendation for timing of aortic valve intervention based on the subject's risk trajectory cluster and predicted clinical outcomes. In some aspects, the analysis module is further configured to provide a risk score for adverse outcomes following transcatheter aortic valve replacement, including mortality, need for permanent pacemaker, and length of hospital stay. In some aspects, the analysis module is further configured to provide a risk score for future onset of aortic stenosis in subjects with false-positive screening results, and wherein the risk score is used to guide longitudinal surveillance.

These aspects and other features and advantages of the invention are described below in more detail.

### Terminology

As used herein, the term “aortic valve stenosis” refers to a thickening and narrowing of the valve between the heart's main pumping chamber and the body's main artery, called the aorta. The narrowing creates a smaller opening for blood to pass through. This reduces or blocks blood flow from the heart to the rest of the body. Traditionally, the severity of AVS is typically assessed based on the degree of valve obstruction, measured using imaging modalities such as echocardiography or cardiac catheterization.

As used herein, the term “control group” refers to one or more of: i) a group of subjects who had undergone both an echocardiogram (Echo) and an electrocardiogram (ECG) within a 180-day window around the time of AVS diagnosis; ii) a group of subjects who may or may not have hypertension, but that underwent an electrocardiogram (ECG) within a 180-day window around the time of AVS diagnosis; and iii) a group of subjects who have undergone an echocardiogram within a 180-day window around the time of AVS diagnosis, but do not have hypertension. Patients with pacemakers were excluded from the definition of control group, as were those diagnosed with valvular heart diseases, amyloidosis, cardiomyopathy, chronic kidney disease (CKD), heart failure, and inflammatory heart diseases.

As used herein, electrocardiogramacess of producing an electrocardiogram (ECG or EKG), a recording of the heart's electrical activity through repeated cardiac cycles.

As used herein, “ECG” generally means a 12-lead ECG taken from a subject while lying down. ECG terminology has two meanings for the word “lead”: 1) the cable used to connect an electrode to the ECG recorder; and 2) the electrical view of the heart obtained from any one combination of electrodes. A standard ECG uses 10 cables to obtain 12 electrical views of the heart. The different views reflect the angles at which electrodes “look” at the heart and the direction of the heart's electrical depolarization. The electrical activity detected by the electrocardiogram machine is measured in millivolts. ECG machines are calibrated so that a raw signal with an amplitude of 1 mV moves the recording stylus vertically 1 cm. A 12-lead ECG consists of three bipolar limb leads (I, II, and III) (further defined below), the unipolar limb leads (AVR, AVL, and AVF), and six unipolar chest leads, also called precordial or V leads, (V₁, V₂, V₃, V₄, V₅, and V₆).

As used herein, the expression “limb leads” refers to three bipolar leads and three unipolar leads obtained from three electrodes attached to the left arm, the right arm, and the left leg, respectively. They can be abbreviated limb leads I, II, III, IV, V, and VI.

As used herein, the “bipolar limb” or “bipolar limb lead” refers to the potential difference between two of the three limb electrodes (I, II, and III).

As used herein, in some instances, the term “Lead I ECG signals” or “Lead I signals” generally refer to the potential difference between electrodes in the right arm-left arm. It is specifically contemplated that the term “Lead I ECG signal” encompasses intermittent single-lead (Lead I) ECG measurements obtained from a wrist-worn device (“wrist-pulse Lead I ECG signal”).

As used herein, the term “Lead II ECG signals” or “Lead II signals” refers to the potential difference between electrodes in the right arm-left leg.

As used herein, the term “Lead III ECG signals” or “Lead III signals” refers to the potential difference between electrodes in the left leg-left arm.

As used herein, “unipolar limb lead” refers to unipolar limb leads IV, V, and VI (AVR, AVL, and AVF).

As used herein, “unipolar chest leads”, “precordial leads” or “V leads” refers to V leads, (V₁, V₂, V₃, V₄, V₅, and V₆).

As used herein, the term “P wave” is a small deflection wave that represents atrial depolarization.

As used herein, the term “PR interval” or “PRI interval” is the time between the first deflection of the P wave and the first deflection of the QRS complex.

As used herein, the term “QRS wave complex” refers to three waves of the QRS complex representing ventricular depolarization: if a wave immediately after the P wave is an upward deflection, it is an R wave; if it is a downward deflection, it is a Q wave. Small Q waves correspond to depolarization of the interventricular septum. Q waves can also relate to breathing and are generally small and thin. They can also signal an old myocardial infarction (in which case they are big and wide). The R wave reflects depolarization of the main mass of the ventricles-hence it is frequently the largest wave. The S wave signifies the final depolarization of the ventricles, at the base of the heart.

As used herein, the term “ST segment” or “ST interval”, is the time between the end of the QRS complex and the start of the T wave. It reflects the period of zero potential between ventricular depolarization and repolarization.

As used herein, the term “T wave” represents ventricular repolarization (atrial repolarization). This is generally obscured by the large QRS complex wave.

As used herein, the term “P_A_I” refers to P wave amplitude of lead I.

As used herein, the term “P_A_II” refers to P wave amplitude of lead II.

As used herein, the term “P_A_III” refers to P wave amplitude of lead III.

As used herein, the term “P_A_V1” refers to P wave amplitude of lead V1.

As used herein, the term “P_A_V2” refers to P wave amplitude of lead V2.

As used herein, the term “P_A_V3” refers to P wave amplitude of lead V3.

As used herein, the term “P_A_V4” refers to P wave amplitude of lead V4.

As used herein, the term “P_A_V5” refers to P wave amplitude of lead V5.

As used herein, the term “P_A_V6” refers to P wave amplitude of lead V6.

As used herein, the term “P_A_aVF” refers to P wave amplitude of lead aVF.

As used herein, the term “P_A_aVR” refers to P wave amplitude of lead aVR.

As used herein, the term “P_A_aVL” refers to P wave amplitude of lead aVL.

As used herein, the term “R_A_I” refers to R wave amplitude of lead I.

As used herein, the term “R_A_II” refers to R wave amplitude of lead II.

As used herein, the term “R_A_III” refers to R wave amplitude of lead III.

As used herein, the term “R_A_V1” refers to R wave amplitude of lead V1.

As used herein, the term “R_A_V2” refers to R wave amplitude of lead V2.

As used herein, the term “R_A_V3” refers to R wave amplitude of lead V3.

As used herein, the term “R_A_V4” refers to R wave amplitude of lead V4.

As used herein, the term “R_A_V5” refers to R wave amplitude of lead V5.

As used herein, the term “R_A_V6” refers to R wave amplitude of lead V6.

As used herein, the term “R_A_aVF” refers to R wave amplitude of lead aVF.

As used herein, the term “R_A_aVR” refers to R wave amplitude of lead aVR.

As used herein, the term “R_A_aVL” refers to R wave amplitude of lead aVL.

As used herein, the term “R_D_I” refers to R wave duration of lead I.

As used herein, the term “R_D_II” refers to R wave duration of lead II.

As used herein, the term “R_D_III” refers to R wave duration of lead III.

As used herein, the term “R_D_V1” refers to R wave duration of lead V1.

As used herein, the term “R_D_V2” refers to R wave duration of lead V2.

As used herein, the term “R_D_V3” refers to R wave duration of lead V3.

As used herein, the term “R_D V4” refers to R wave duration of lead V4.

As used herein, the term “R_D_V5” refers to R wave duration of lead V5.

As used herein, the term “R_A_V6” refers to R wave amplitude in lead V6.

As used herein, the term “R_A_aVF” refers to R wave amplitude of lead aVF.

As used herein, the term “R_D_I” refers to R wave duration of lead I.

As used herein, the term “R_D_II” refers to R wave duration of lead II.

As used herein, the term “R_D_III” refers to R wave duration of lead III.

As used herein, the term “R_D_V1” refers to R wave duration of lead V1.

As used herein, the term “R_D_V2” refers to R wave duration of lead V2.

As used herein, the term “R_D_V3” refers to R wave duration of lead V3.

As used herein, the term “R_D_V4” refers to R wave duration of lead V4.

As used herein, the term “R_D_V5” refers to R wave duration of lead V5.

As used herein, the term “R_D_V6” refers to R wave duration of lead V6.

As used herein, the term “R_D_aVF” refers to R wave duration of lead a VF.

As used herein, the term “R_D_aVR” refers to R wave duration of lead a VR.

As used herein, the term “R_D_aVL” refers to R wave duration of lead aVL.

As used herein, the term “S_A_I” refers to S wave amplitude of lead I.

As used herein, the term “S_A_II” refers to S wave amplitude of lead II.

As used herein, the term “S_A_III” refers to S wave amplitude of lead III.

As used herein, the term “S_A_V1” refers to S wave amplitude of lead V1.

As used herein, the term “S_A_V2” refers to S wave amplitude of lead V2.

As used herein, the term “S_A_V3” refers to S wave amplitude of lead V3.

As used herein, the term “S_A_V4” refers to S wave amplitude of lead V4.

As used herein, the term “S_A_V5” refers to S wave amplitude of lead V5.

As used herein, the term “S_A_V6” refers to S wave amplitude of lead V6.

As used herein, the term “S_A_aVF” refers to S wave amplitude of lead aVF.

As used herein, the term “S_A_aVR” refers to S wave amplitude of lead aVR.

As used herein, the term “S_A_aVL” refers to S wave amplitude of lead aVL.

As used herein, the term “T_A_I” refers to T wave amplitude of lead I.

As used herein, the term “T_A_II” refers to T wave amplitude of lead II.

As used herein, the term “T_A_III” refers to T wave amplitude of lead III.

As used herein, the term “T_A_V1” refers to T wave amplitude of lead V1.

As used herein, the term “T_A_V2” refers to T wave amplitude of lead V2.

As used herein, the term “T_A_V3” refers to T wave amplitude of lead V3.

As used herein, the term “T_A_V4” refers to T wave amplitude of lead V4.

As used herein, the term “T_A_V5” refers to T wave amplitude of lead V5.

As used herein, the term “T_A_V6” refers to T wave amplitude of lead V6.

As used herein, the term “T_A_aVF” refers to T wave amplitude of lead aVF.

As used herein, the term “T_A_aVR” refers to T wave amplitude of lead a VR.

As used herein, the term “T_A_aVL” refers to T wave amplitude of lead aVL.

As used herein, the term “QT” refers to the QT interval, also referred to as the QT value.

As used herein, the term “true positive” or “TP” refers to an unactionable event correctly identified as “low” quality.

As used herein, the term “false positive” or “FP” refers to an unactionable event correctly identified as “high” quality.

As used herein, the term “true negative” or “TN” refers to an actionable event correctly identified as “high” quality.

As used herein, the term “false negative” or “FN” refers to an actionable event incorrectly classified as “low” quality.

As used herein, the term “sensitivity”, abbreviated “Se”, refers to the ability to correctly classify unactionable data as “low” quality.

As used herein, the term “positive predictive value”, abbreviated “PPV”, refers to the probability that data classified as “low” quality is unactionable.

As used herein, the term “about” and the term “approximately,” when used to modify a numeric value, indicate that deviations of up to 10% above and below the numeric value remain within the intended meaning of the recited value.

Designation of a range of values includes all integers within or defining the range, and all subranges defined by integers within the range.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other features and advantages of the present invention will be more fully understood from the following detailed description of illustrative configurations taken in conjunction with the accompanying drawings in which:

FIG. 1 (FIG. 1) is a flow chart with illustrative steps for curating the data for model development.

FIG. 2 (FIG. 2) is a flow chart with illustrative steps of the feed-forward neural network deployed in developing the model.

FIG. 3 (FIG. 3) is an illustration of the SHAP (SHapley Additive explanations) depicting the influence of different features on the model's predictive ability to identify aortic stenosis. It was observed that variables such as AGE and QT interval exerted a substantial impact across the dataset, showcasing a remarkable degree of influence on the predictive outcomes.

FIG. 4 (FIG. 4) is a bee swarm diagram below showcasing predictive SHAP values derived from a unified model.

FIG. 5 (FIG. 5) is a flow chart depicting steps for the creation of the validation dataset.

FIG. 6 (FIG. 6) is an AUC chart indicating area under the receiver operating characteristic curve for the case study test cases.

FIGS. 7A-7D (FIGS. 7a-7D) are charts depicting the validation of systems and processes of the disclosure in predicting moderate/severe AS and risk of incident HF in a study of 3,662 participants. FIG. 7A is the median model scores by aortic stenosis grade/severity categories. FIG. 7B is an ROC curve analysis yielding an AUROC of 0.79 for predicting moderate/severe aortic stenosis. FIG. 7C probability of moderate/severe aortic stenosis over time based on positive vs. negative initial model screening result. FIG. 7D estimated 5-year probability of moderate/severe aortic stenosis progression stratified by initial positive vs. negative model screening result.

FIG. 8 (FIG. 8) is an illustration of an exemplary output of the analysis provided by the systems and processes of the disclosure.

FIG. 9 (FIG. 9) is a schematic of a study design used to evaluate the system and processes of the disclosure on a population of 1,596 patients before and after TAVR.

FIG. 10A (FIG. 10A) is a graph showing a breakdown of a retrospective study of 7,860 ECGs from 2,040 TAVR recipients collected up to 10 years pre-procedure. FIG. 10A displays outputs of the system and its processes (i.e. a score) as a function of years before TAVR.

FIG. 10B (FIG. 10B) is a graph showing a breakdown of a retrospective study displaying All cause mortality (%) as a function of time after TAVR (months). The data is broken down into three distinct clusters, stable low, accelerated progression, and persistently high.

FIG. 11A-B (FIG. 11A) are graphs illustrating that an output of the systems and processes described herein provide independent information for predicting 5-year mortality. FIG. 11A illustrates an application of the model to 7,860 ECGs from 2,040 patients, and it displays scores grouped into three trajectory clusters: Low, Steep Increase, and High. FIG. 11B displays associations between trajectory clusters and 1-year mortality, 5-year mortality.

It should be understood that the drawings are not necessarily to scale (e.g., schematics), and that like reference numbers refer to like features.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### I. Overview

All of the functionalities described in connection with one embodiment of the methods, compositions, or formulations described herein are intended to be applicable to the additional configurations of the methods, compositions, or formulations described herein except where expressly stated or where the feature or function is incompatible with the additional configurations. For example, where a given feature or function of component is expressly described in connection with one embodiment but not expressly mentioned in connection with an alternative embodiment, it should be understood that the feature or component may be deployed, utilized, or implemented in connection with the alternative embodiment unless the feature or component is incompatible with the alternative embodiment.

Aortic valve stenosis (AVS), or Aortic stenosis (AS), is a cardiovascular condition characterized by the narrowing of the aortic valve opening, which obstructs the flow of blood from the left ventricle to the aorta, thereby impeding the efficient delivery of oxygenated blood to the body. This narrowing occurs due to the thickening and calcification of the valve leaflets, leading to reduced cardiac output and potentially life-threatening complications.

AVS is prevalent among the elderly population, particularly those over the age of 65, with its incidence increasing significantly with advancing age. It is estimated that approximately 2-5% of individuals over 65 years old have some degree of aortic valve stenosis. However, AVS can also occur in younger individuals, albeit less frequently, due to congenital abnormalities, rheumatic fever, or other underlying medical conditions.

The severity of AVS is typically assessed based on the degree of valve obstruction, measured using imaging modalities such as echocardiography or cardiac catheterization. Mild stenosis may be asymptomatic and require only close monitoring, while severe stenosis can result in symptoms such as chest pain, dyspnea, and syncope, indicating the need for intervention. Treatment options for AVS include medication to manage symptoms, such as diuretics and beta-blockers, as well as surgical interventions like aortic valve replacement (AVR) or transcatheter aortic valve replacement (TAVR) for severe cases.

Early detection and intervention are paramount in managing cardiovascular conditions like aortic valve stenosis (AVS). Electrocardiogram (ECG) can be an important screening tool, especially in regions where access to echocardiography, the gold standard for AVS diagnosis, may be limited. Through ECG, healthcare providers could swiftly identify individuals exhibiting characteristic patterns associated with AVS, enabling targeted assessment and prompt initiation of appropriate management strategies. These patterns can include indicators of left ventricular hypertrophy (LVH), left atrial enlargement (LAE), and various conduction abnormalities. LVH, for instance, may reflects the heart's response to pressure overload due to aortic valve narrowing, serving as an early sign of potential AVS. Likewise, LAE and conduction abnormalities on ECG signify the cardiac remodeling and electrical disturbances commonly observed in AVS patients. Recognizing these patterns prompts further diagnostic evaluations, facilitating timely interventions and optimizing patient outcomes.

Integrating ECG screening into routine clinical practice offers significant benefits, particularly in resource-constrained settings. ECG is widely available, cost-effective, and non-invasive, making it a pragmatic option for large-scale screening efforts. Its simplicity and accessibility enhance its utility in identifying individuals at risk of AVS, enabling healthcare providers to allocate resources efficiently and prioritize high-risk patients for further evaluation. By leveraging ECG as a frontline screening tool, healthcare systems can streamline diagnostic pathways, mitigate delays in AVS detection, and expedite appropriate interventions, thereby potentially reducing disease burden and improving patient prognosis.

The present disclosure describes the development and validation of a process for screening for AVS utilizing input from a traditional 12-lead ECG, which provided systems having analysis modules for analyzing the inputs. The process of the disclosure implements certain steps of a process where a database has been carefully selected and curated to associate certain ECG parameters with diagnosis of AVS. In the process, an artificial intelligence model, specifically trained and curated for identifying the presence of Aortic Valve Stenosis (AVS) features by analyzing parameters from all 12 leads and the age of the patient, is applied to the analysis of parameters to 12-lead ECG signals. The disclosure describes a carefully curated and validated measurement matrix, trained to discern distinctive patterns and characteristics that serve as strong indicators for the detection of AVS. The measurement matrix comprises a plurality of ECG parameters or ECG measurements, which encapsulate essential information regarding the cardiac signal's characteristics and attributes. The process described herein not only provided a high predictive value for AVS, but also identified unexpected ECG parameters that support the detection of AVS from ECG signals.

In some aspects, the method involves screening a plurality of parameters from one or more ECG leads for a pattern predictive of aortic valve stenosis. The screening is performed by a computer program model trained to identify the predictive pattern from the plurality of parameters, i.e., a computer program with an analysis module trained to identify aortic stenosis patterns. In many instances, at least a first parameter is the age of the subject associated with the ECG and at least a second parameter is selected from one or more of the following: P wave amplitude, R wave amplitude, R wave duration, S wave amplitude, S wave duration, T wave amplitude, and QT interval. The method outputs a value that is predictive of aortic valve stenosis based on the identified pattern.

At least one system described herein comprises an analysis module whereby the analysis module identifies patterns such as a reduced QT interval as a function of the subject's age, increased T amplitude from specific leads, and other combinations of parameters. In some cases, the analysis module was developed to consider performance metrics for a negative predictive value greater than 90%, sensitivity greater than 70%, and specificity greater than 70%. The analysis module can be a feedforward neural network trained on 62 parameters, including age, P wave amplitude, R wave amplitude, R wave duration, S wave amplitude, S wave duration, T wave amplitude, and average QT.

In some aspects, the disclosed system leverages AI algorithms with modules devised and trained on large, diverse datasets to analyze 12-lead ECGs and generate a risk score for moderate-to-severe AS. The system can be integrated into ECG devices at the point of care, hospital EHRs, and similar to enable automated, population-level screening using existing ECG data. The model provides both diagnostic and prognostic outputs, including the likelihood of current AS, risk of future AS or heart failure, and post-intervention outcomes.

In some aspects, the system comprises an input module (e.g., a computer module) for receiving ECG data, an analysis module (e.g., a computer module) for implementing a feedforward neural network trained on up to 62 ECG parameters and patient age, and an output module (e.g., a computer module trained with AI) providing risk and prognostic scores. The model is trained and validated on datasets with confirmed AS status by echocardiography, excluding confounding conditions such as pacemaker, prior MI, or LVH. The analysis module is specifically devised and engineered to consider select inputs, e.g., at least an age of a subject associated with an ECG and a QT interval of the subject.

The model and systems disclosed herein not only accurately predict prevalent moderate/severe AS but also identify individuals at increased risk for future AS and HF, supporting its use as, e.g., a community screening tool for early intervention.

Further detail is provided below.

### II. Processes for Distinguishing Aortic Valve Stenosis

Aortic stenosis (AS) is one of the most common and serious valve disease problems. The heart pumps blood through the aortic valve to the body. Over time, calcium buildup can narrow the valve opening and restrict blood flow to the heart. If left undiagnosed or untreated in a timely fashion, it can become more severe and can ultimately lead to heart failure and death.

An electrocardiogram (ECG or EKG) is one of the simplest and fastest tests used to evaluate the heart. Electrodes (small, plastic patches that stick to the skin) are placed at certain spots on the chest, arms, and legs. The electrodes are connected to an ECG machine by lead wires. The electrical activity of the heart is then measured and reported. To date however, there is no reliable, reproducible, process or system that has been successfully applied to translating the electric signals from an ECG into a diagnosis of aortic valve stenosis. Unfortunately, the diagnosis of aortic stenosis is made mostly on physical examination and by echocardiography. The present disclosure provides a solution to the existing challenges, by providing a process and systems that can detect aortic valve stenosis accurately and reliably from an ECG.

In some aspects the disclosure provides a process for screening for aortic valve stenosis from one or more, two or more, three or more, four or more, five or more, six or more, seven or more, eight or more, nine or more, ten or more, eleven or more, or from parameters from all 12-electrocardiogram (ECG) leads. Typically, an ECG instrument records each lead separately, either sequentially or, in some instruments, several leads can be recorded simultaneously. As the stylus moves, depending on the voltage it is reflecting, the recording paper moves at a constant, present speed generally of 25 mm/sec. Hence time is represented on the recording paper by the horizontal axis, and voltage is reflected in the vertical axis. The signal is recorded on a grid, with lines 1 mm apart in both the vertical and horizontal axes. In the horizontal axis, each 1 mm generally represents 0.04 second (40 msec), and every 5 mm, designated by a bold line, indicates 0.2 second. The recording is generally standardized, so that 1 mm vertical deflection reflects 0.1 mV; 5 mm, again indicated by a more bold line, represents 0.5 mV. If the electrocardiogram is recorded at a different paper speed (such as twice the conventional rate) or with a voltage other than the conventional, these alterations are usually recorded and taken into account when measuring the various intervals and waves of the ECG.

By convention, the first upward deflection from the baseline is termed the P wave, and it reflects atrial depolarization. It is understood that in healthy scenarios, the P wave should not exceed 2.5 mm in height nor 0.11 second in width (i.e., less than three small boxes high and wide). Ventricular depolarization is represented by the QRS complex. The Q wave is the first negative deflection from the baseline after the P wave, but preceding an upward deflection. Normally, the Q wave reflects ventricular septal depolarization, and its duration does not exceed 0.03 second. The R wave is the first positive deflection after the P wave, reflecting depolarization of the ventricular mass. The S wave is the negative deflection following the positive R wave representing later ventricular depolarization. Any positive deflection following an S wave is labeled R′ (“R-prime”); any negative deflection following an R′ is labeled S′. By convention, an uppercase R or S infers a large deflection, whereas a lowercase r or s infers a smaller deflection. The T wave reflects repolarization of the ventricle and may be represented as either a positive or negative deflection following the QRS complex. The area incorporated within the T wave approximates that within the QRS complex, and its polarity is roughly the same as the principal QRS polarity. Occasionally, another wave, the U wave, may follow the T wave, and it is generally of the same polarity as the T wave. The mechanism of the U wave is unknown, though it may reflect repolarization of papillary muscles, or simply represent an afterpotential. The PR interval is the time from the beginning of the P wave to the beginning of the QRS, whether initiated by a Q or an R, and this interval indicates the time required for the atria to depolarize, and for the electrical current to conduct through the atrioventricular node and bundle branches until the ventricle depolarizes. The QRS interval is that interval from the beginning of the Q wave to the end of the S wave, incorporating ventricular depolarization. The QT interval is the time from the beginning of the Q wave to the end of the T wave, incorporating both ventricular depolarization and repolarization. The PR segment is that portion of the recording between the end of the P wave and the beginning of the QRS. The ST segment is that portion of the recording, generally represented by a horizontal line, from the end of ventricular depolarization, whether represented by an R wave or an S wave, to the beginning of the T wave.

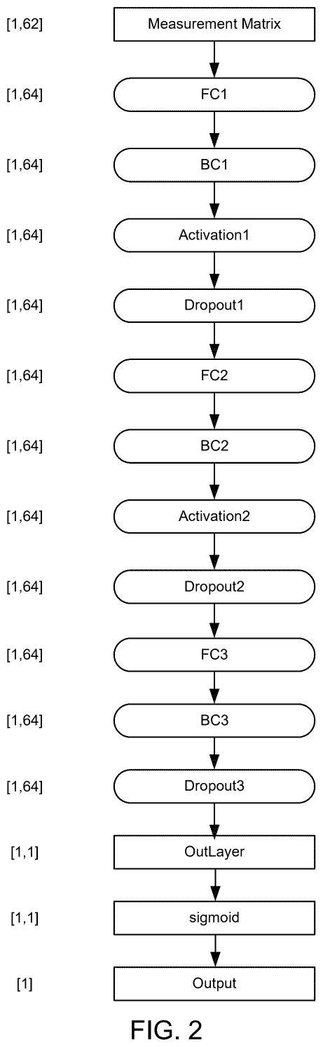

In the instant disclosure Applicant describes a novel model architecture carefully selected to measurement matrices of 62 parameters and produces a parameter output between 0 and 1 (see FIG. 2). In the instantly described systems, it was observed that variables such as AGE and QT interval exerted a substantial impact across the dataset, showcasing a remarkable degree of influence on the predictive outcomes. These findings underscore the importance of considering the QT interval and age in assessing predictive models' performance and reliability. The robust influence of age highlights the significance of age-related physiological changes in AVS, whereas the QT interval's prominence underscores its clinical relevance as a measure of ventricular depolarization and repolarization. This was particular noteworthy it neither age not QT interval were found to have any significant effect when Applicant developed models for the detection of hyperkalemia (see, U.S. provisional patent application 63/694,511) or when Applicant developed models for the detection of cardiac amyloidosis (see, U.S. provisional patent application 63/610,373).

The disclosure describes a novel processes and systems for assessing ECG patterns for the presence of characteristics indicative of aortic valve stenosis consisting of assessments of one or more wave amplitude(s), wave duration, and interval length. In some aspects the disclosure provides a method for identifying one or more electrocardiogram (ECG) parameters(s) that are informative of aortic valve stenosis, comprising: annotating a plurality of electrocardiogram (ECG) parameters in a database as associated with one or more aortic valve stenosis designations or a negative aortic valve stenosis control designation, whereby the designation for the aortic valve stenosis is selected from the group consisting of: rheumatic disorder of both mitral and aortic valve, nonrheumatic AVS, nonrheumatic AVS with insufficiency, congenital stenosis of AVS, bicuspid aortic valve, congenital insufficiency of aortic valve, aortic insufficiency/stenosis, nonrheumatic aortic valve disorder, unspecified other nonrheumatic AVS, stenosis aortic moderate, stenosis aortic congenital, regurgitation aortic, rheumatic aortic stenosis with insufficiency, congenital subaortic stenosis, supravalvular aortic stenosis, rheumatic aortic stenosis with insufficiency, stenosis aortic rheumatic, aortic valve disease, aortic regurgitation, and rheumatic aortic stenosis; instructing a machine learning model to distinguish the plurality of electrocardiogram (ECG) parameters, wherein the machine learning model is instructed to distinguish one or more parameters selected from a group comprising a P wave duration, a P wave amplitude, an R wave duration, an R wave amplitude, an S wave amplitude, a T wave duration, a T wave amplitude, a PR Interval (PRI) value, and a QT value based on a pattern present in the one or more AVS designation(s) that is not present in the negative control; applying a SHAP analysis to the plurality of distinguished electrocardiogram (ECG) parameters thereby providing a numeric value that represents the contribution of each parameter to the one or more aortic valve stenosis designation and identifies one or more electrocardiogram (ECG) parameters(s) that are informative of AVS. In many instances, and to establish a comparative framework for the study, three control groups were chosen. The first control group individuals did not undergo an echocardiogram. The second control group includes subjects that may or may not have hypertension, but those subjects with a pacemaker are excluded to maintain consistency with the other groups. Third control group consist of participants who have undergone an echocardiogram but do not have hypertension.

The P wave duration can correspond, e.g., to the P wave duration in lead I, the P wave duration in lead II, the P wave duration in lead III, the P wave duration in lead V₁, the P wave duration in lead V₂, the P wave duration in lead V₃, the P wave duration in lead V₄, the P wave duration in lead V₅, the P wave duration in lead V₆, the P wave duration in lead aVF, the P wave duration in lead aVR, or the P wave duration in lead aVL. The P wave amplitude can correspond to, e.g., the P wave amplitude in lead I, the P wave amplitude in lead II, the P wave amplitude in lead III, the P wave amplitude in lead V₁, the P wave amplitude in lead V₂, the P wave amplitude in lead V₃, the P wave amplitude in lead V₄, the P wave amplitude in lead V₅, the P wave amplitude in lead V₆, the P wave amplitude in lead aVF, the P wave amplitude in lead a VR, or the P wave amplitude in lead aVL. The R wave duration can correspond, e.g., to the R wave duration in lead I, the R wave duration in lead II, the R wave duration in lead III, the R wave duration in lead V₁, the R wave duration in lead V₂, the R wave duration in lead V₃, the R wave duration in lead V₄, the R wave duration in lead V₅, the R wave duration in lead V₆, the R wave duration in lead aVF, the R wave duration in lead aVR, or the R wave duration in lead aVL. The R wave amplitude corresponds to, e.g., the R wave amplitude in lead I, the R wave amplitude in lead II, the R wave amplitude in lead III, the R wave amplitude in lead V₁, the R wave amplitude in lead V₂, the R wave amplitude in lead V₃, the R wave amplitude in lead V₄, the R wave amplitude in lead V₅, or the R wave amplitude in lead V₆, the R wave amplitude in lead a VF, the R wave amplitude in lead aVR, or the R wave amplitude in lead aVL. The S wave amplitude can correspond to, e.g., the S wave amplitude in lead I, the S wave amplitude in lead II, the S wave amplitude in lead III, the S wave amplitude corresponds to the S wave amplitude in lead V₁, the S wave amplitude in lead V₂, the S wave amplitude in lead V₃, the S wave amplitude in lead V₄, the S wave amplitude in lead V₅, the S wave amplitude in lead V₆, the S wave amplitude in lead aVF, the S wave amplitude in lead aVR, or the S wave amplitude in lead aVL. The T wave amplitude corresponds to, e.g., the T wave amplitude in lead I, the T wave amplitude in lead II, the T wave amplitude in lead III, the T wave amplitude in lead V₁, the T wave amplitude in lead V₂, the T wave amplitude in lead V₃, the T wave amplitude in lead V₄, the T wave amplitude in lead V₅, the T wave amplitude in lead V₆, the T wave amplitude in lead aVF, the T wave amplitude in lead aVR, or the T wave amplitude in lead aVL. The PR interval can correspond to, e.g., a value in lead I, a value in lead II, a value in lead III, a value in lead V₁, a value in lead V₂, a value in lead V₃, a value in lead V₄, a value in lead V₅, a value in lead V₆, a value in lead aVF, wherein the PR interval is a value in lead aVL, or a value in lead aVR. In some instances, the machine learning model is a feed forward model, such as the model demonstrated to identify aortic valve stenosis patterns in the Examples.

In some instances, the machine learning model is instructed to consider at least 1 feature, at least 2 features, at least 3 features, at least 4 features, at least 5 features, at least 6 features, at least 7 features, at least 8 features, at least 9 features, at least 10 features, at least 11 features, at least 12 features, at least 13 features, at least 14 features, at least 15 features, at least 16 features, at least 17 features, at least 18 features, at least 19 features, at least 20 features, at least 21 features, at least 22 features, at least 23 features, at least 24 features, at least 25 features, at least 26 features, at least 27 features, at least 28 features, at least 29 features, at least 30 features, at least 31 features, at least 32 features, at least 33 features, at least 34 features, at least 35 features, at least 36 features, at least 37 features, at least 38 features, at least 39 features, at least 40 features, at least 41 features, at least 42 features, at least 43 features, at least 44 features, at least 45 features, at least 46 features, at least 47 features, at least 48 features, at least 49 features, at least 50 features, at least 51 features, at least 52 features, at least 53 features, at least 54 features, at least 55 features, at least 56 features, at least 57 features, at least 58 features, at least 59 features, at least 60 features, at least 61 features, or at least 62 features from parameters of lead ECG signals. In some instances, the machine learning model is instructed to distinguish 62 ECG parameters and an age parameter. In some instances, the machine learning model receives measurement matrices of 62 ECG parameters from the database and produces a parameter output between 0 and 1. In specific instances, the machine learning model is instructed to distinguish 62 ECG parameters, specifically the machine learning model receives measurement matrices of one or more of the 62 aforementioned parameters from the database and produces a parameter output between 0 and 1.

The disclosure describes a process in which, through careful conception of input ECG parameters and annotation of a database with information that it does not inherently possess-nor is it able to provide on its own-deep analysis of all 12 parameters of an ECG to discern distinctive patterns and characteristics that serve as strong indicators for the detection of AVS. A system of the disclosure harnessed information embedded within the measurement matrix (see Examples) and gained a deep understanding of the data derived from the age and parameters of all the 12 leads of a plurality of ECG signals. The dataset encompassed electrocardiograms (ECGs) from a cohort of millions of subjects. Each patient's data profile is characterized by 8 distinct waveforms, aligning with the 8 different leads: I, II, V₁, V₂, V₃, V₄, V₅, and V₆, originally encoded in the base64 format. Each waveform extends across a time span of approximately 10 seconds operating at either 250 or 500 samples per second. The database was annotated a diagnosis code indicative of any form of AVS, and a subset of approximately 3000 patients was used for training. Parameters were trained for binary classification. The model's compilation was configured to use binary cross-entropy loss, the Adam optimizer, and accuracy as the evaluation metric and incorporated two callbacks, one to prevent overfitting by monitoring validation metrics; the other to adjust learning rate.

The disclosure describes a process in which, e.g., automated screening of existing ECG devices at the point of care, ECGs in EHRs, and similar can be implemented to identify undiagnosed moderate-to-severe AS. The disclosure demonstrates the predictive and the prognostics capabilities of the analysis: a) predictive analytics: the system and processes described herein provides a risk score indicating the probability of current or future AS and heart failure; b) prognostic value: score trajectories and periprocedural changes predict mortality, need for pacemaker, and length of hospital stay post-TAVR.

### QT Interval (Duration), QTc Interval, the QRS Complex and its Components, the Q, R, and S Waves in the Assessment of AVS

QT duration reflects the total duration of ventricular depolarization and repolarization. It is measured from the onset of the QRS complex to the end of the T-wave. The QT duration is inversely related to the heart rate; i.e., the QT interval increases at slower heart rates and decreases at higher heart rates. Therefore, typically, the art teaches that to determine whether the QT interval is within normal limits, it is necessary to adjust for the heart rate. The heart rate-adjusted QT interval is referred to as the corrected QT interval (QTc interval).

Generally, the QRS complex represents depolarization (activation) of the ventricles. It is generally referred to as the “QRS complex” although it may not always display all three waves. Since the electrical vector generated by the left ventricle is many times larger than the vector generated by the right ventricle, the QRS complex is actually a reflection of left ventricular depolarization, factors that make the analysis of a QRS signal complex. In the context of disease, for instance, an R wave or an S wave may not produce standard or readily recognizable signals. QRS duration is the time interval from the onset to end of the QRS complex. Typically, it is understood that a short QRS complex is desirable as it proves that the ventricles are depolarized rapidly, which in turn implies that that the conduction system functions properly. In contrast, wide (also referred to as broad) QRS complexes indicate that ventricular depolarization is slow, which may be due to dysfunction in the conduction system.

The disclosure demonstrates that, in the context of aortic valve stenosis, as the QT interval decreases, distinct patterns emerge in the impact of various features on the model's predictions, shedding light on their dynamic interplay. Notably, the T amplitude from lead V4 (T A v4) exhibits a notable increase, suggesting that lower QT intervals are associated with higher values of T amplitude in this specific lead. This finding underscores the potential role of the QT interval in modulating the amplitude of T waves in certain electrocardiographic leads, which could have clinical implications for identifying cardiac abnormalities. As illustrated herein, at least one model were created where the QT interval and the age produce the most informative signals for detection of aortic stenosis.

### The P-Wave, PR Interval and PR Segments

Generally, ECG interpretation traditionally starts with an assessment of the P-wave, which reflects atrial depolarization. The PR interval is the distance between the onset of the P-wave to the onset of the QRS complex. The PR interval typically provides information on whether impulse conduction through the atrioventricular node falls under known standards. The PR segments serves as the baseline (also referred to as the reference line or isoelectric line) of the ECG curve. The amplitude is measured by using the PR segment as the baseline.

### The J Point and the ST Segment

The ST segment corresponds to the plateau phase (phase 2) of the action potential. The ST segment is reported to be altered in a wide range of conditions, producing characteristic ST segment changes (e.g., ischemia). There are two types of ST segment deviations. ST segment depression implies that the ST segment is displaced, such that it is below the level of the PR segment. ST segment elevation implies that the ST segment is displaced, such that it is above the level of the PR segment. The magnitude of depression/elevation is measured as the height difference (in millimeters) between the J point and the PR segment. The J point is the point where the ST segment starts. If the baseline (PR segment) is difficult to discern, the TP interval may be used as the reference level.

### The T-Wave

The T-wave reflects the rapid repolarization of contractile cells and T-wave changes occur in a wide range of conditions. T-wave changes are frequently misunderstood in clinical practice, which the discussion below will attempt to cure. The transition from the ST segment to the T-wave should be smooth (and not abrupt). The normal T-wave is slightly asymmetric, with a steeper downward slope.

### The U-Wave

The U-wave is seen occasionally. It is a positive wave occurring after the T-wave. Its amplitude is generally one-fourth of the T-wave's amplitude. The U-wave is most frequently seen in leads V₂-V₄. Individuals with prominent T-waves, as well as those with slow heart rates, display U-waves more often. The genesis of the U-wave remains elusive.

Such methods and systems identified novel parameters associated with AVS and provide methods and systems for detection of AVS with a sensitivity greater than 90%, greater than 91%, greater than 92%, greater than 93%, greater than 94%, greater than 95%, greater than 96%, greater than 97%, greater than 98%, and in at least one model with a sensitivity greater than 99%. Similarly, the methods and systems described herein identified novel parameters associated with aortic valve stenosis and provide methods and systems for detection of aortic valve stenosis with a specificity greater than 90%, greater than 91%, greater than 92%, greater than 93%, greater than 94%, greater than 95%, greater than 96%, greater than 97%, greater than 98%, and in at least one model with a specificity greater than 99%.

In some aspects, the disclosure provides a process for screening an ECG for a pattern that is predictive of aortic valve stenosis, the process comprising: screening a plurality of parameters from one or more electrocardiogram (ECG) leads for a pattern that is predictive of aortic valve stenosis, whereby the screening is performed by a computer program model trained to identify the predictive pattern from the plurality of parameters, whereby: at least a first parameter in the plurality of parameters is an age of a subject associated with the ECG; and at least a second parameter is selected from one or more of P wave amplitude, R wave amplitude, R wave duration, S wave amplitude, S wave duration, T wave amplitude, and QT interval, an outputting a value that is predictive of aortic valve stenosis based on the pattern that is predictive of aortic valve stenosis. In preferred cases, the at least one second parameter is the QT interval. In some cases, the at least one second parameter is one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, sixty-one or more, or all sixty-two parameters selected from the group consisting of one or more of the QT interval, a T amplitude from lead V4, a T amplitude from lead AVL, a R amplitude from lead II, a P wave amplitude of lead I, a P wave amplitude of lead II, a P wave amplitude of lead III, a P wave amplitude of lead V1, a P wave amplitude of lead V2, a P wave amplitude of lead V3, a P wave amplitude of lead V4, a P wave amplitude of lead V5, a P wave amplitude of lead V6, a P wave amplitude of lead aVF, a P wave amplitude of lead a VR, a P wave amplitude of lead aVL, a R wave amplitude of lead I, a R wave amplitude of lead III, a R wave amplitude of lead V1, a R wave amplitude of lead V2, a R wave amplitude of lead V3, a R wave amplitude of lead V4, a R wave amplitude of lead V5, an R wave amplitude of lead V6, a R wave amplitude of lead aVF, a R wave amplitude of lead aVR, a R wave amplitude of lead aVL, a R wave duration of lead aVL, a R wave duration of lead I, a R wave duration of lead II, a R wave duration of lead III, a R wave duration of lead V1, a R wave duration of lead V2, a R wave duration of lead V3, a R wave duration of lead V4, a R wave duration of lead V5, a R wave duration of lead V6, a R wave duration of lead aVF, a R wave duration of lead aVR, a R wave duration of lead aVL, a S wave amplitude of lead I, a S wave amplitude of lead II, a S wave amplitude of lead III, a S wave amplitude of lead V1, a S wave amplitude of lead V2, a S wave amplitude of lead V3, a S wave amplitude of lead V4, a S wave amplitude of lead V5, a S wave amplitude of lead V6, a S wave amplitude of lead aVF, a S wave amplitude of lead aVR, a S wave amplitude of lead aVL, a T wave amplitude of lead I, a T wave amplitude of lead II, a T wave amplitude of lead III, a T wave amplitude of lead V1, a T wave amplitude of lead V2, a T wave amplitude of lead V3, a T wave amplitude of lead V5, a T wave amplitude of lead V6, a T wave amplitude of lead aVF, a T wave amplitude of lead aVR. In some cases, the disclosure provides a process implementing a computer model, and the pattern that is predictive of aortic valve stenosis in the computer model is a reduced QT interval as a function of the age of the subject associated with the ECG. In some instances, a negative predictive value of the pattern is greater than 90%. In some instances, the aortic valve stenosis is rheumatic disorder of both mitral and aortic valve, nonrheumatic AVS, nonrheumatic AVS with insufficiency, congenital stenosis of AVS, bicuspid aortic valve, congenital insufficiency of aortic valve, aortic insufficiency/stenosis, nonrheumatic aortic valve disorder, unspecified other nonrheumatic AVS, stenosis aortic moderate, stenosis aortic congenital, regurgitation aortic, rheumatic aortic stenosis with insufficiency, congenital subaortic stenosis, supravalvular aortic stenosis, rheumatic aortic stenosis with insufficiency, stenosis aortic rheumatic, aortic valve disease, aortic regurgitation, and rheumatic aortic stenosis. In some instances, in the models disclosed herein, a user directs the training of the model to adjusts the learning rate dynamically based on the plateauing of a monitored metric. In some instances, in the models disclosed herein, a user directs the training of the model to prevent overfitting by monitoring validation metrics.

### III. Systems for Detecting Aortic Stenosis

In some aspects, the disclosure provides systems and processes for identifying aortic valve stenosis. The system disclosed herein, or a computer system used in the analyses of one or more features from various waveforms, can share the results with a third-party from any other facility, such as a hospital a clinical facility or another heath care organization. In some aspects, the disclosure provides a system for screening an ECG for a pattern that is predictive of aortic valve stenosis, the system comprising: an input module for receiving a plurality of parameters from one or more electrocardiogram (ECG) leads; an analysis module comprising a computer program model trained to identify a pattern predictive of aortic valve stenosis from the plurality of parameters from at least two inputs: an age of a subject associated with the ECG; and QT interval, an output module for outputting a predictive of aortic valve stenosis based on the pattern predictive of aortic stenosis from the at least two inputs.

In some configurations, a system of the disclosure comprises the following components:

### Components:

Input Module: Receives a plurality of parameters from one or more ECG leads.

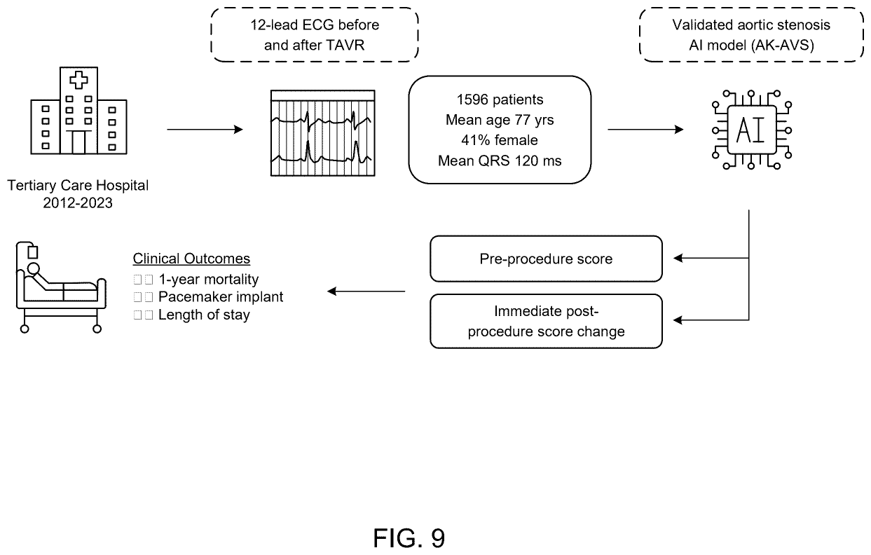

Analysis Module: Comprises a computer program model trained to identify patterns predictive of AVS from the parameters. The analysis module employs a machine learning algorithm, such as a support vector machine (SVM) or random forest, to identify a pattern predictive of aortic valve stenosis from at least two inputs: the age of the subject and the QT interval. The module can also identify patterns from additional inputs (e.g., at least 3 inputs) such as T amplitude from lead V4, T amplitude from lead AVL, and R amplitude from lead II. Further, the SVM uses a radial basis function (RBF) kernel with optimized hyperparameters (gamma=0.1, C=10) achieved through a grid search strategy. The random forest consists of 100 decision trees, each trained on a random subset of the data using the CART algorithm with Gini impurity as the splitting criterion. The analysis module can be programmed as outlined in Example 1, to provide performance metrics that aims for a negative predictive value greater than 90%, sensitivity greater than 70%, and specificity greater than 70%. See Example 1. The output module can outputs a predictive value for AVS based on the identified pattern, in a variety of formats, one such illustrative format is depicted on FIG. 9.

Some systems are configured for detecting, e.g.: a wave duration (e.g., milliseconds, seconds) of a P wave, an R wave, an S wave, a T wave, of one or more of the 12 ECG leads (lead I, lead II, lead III, lead V₁, lead V₂, lead V₃, lead V₄, lead V₅, lead V₆, lead aVF, lead aVR, lead aVL); e.g., a wave amplitude (e.g., 0.1 millimeter to 1 cm and values in between) of a P wave, an R wave, an S wave, a T wave of one or more of the 12 ECG leads (lead I, lead II, lead III, lead V₁, lead V₂, lead V₃, lead V₄, lead V₅, lead V₆, lead aVF, lead aVR, lead aVL); a PR Interval (PRI) value (e.g., milliseconds, seconds), a QT value (e.g., milliseconds, seconds).

A system of the disclosure can comprise a computer operating system configured to perform executable instructions, such as instructions required to, e.g., R-waves, S-waves, P-wave, PR intervals, PR segments, T-wave, U-wave, RR interval, PP interval, ST-T segments, TP interval, QRS duration, R-wave amplitude, S-wave amplitude, QT duration, and other suitable parameters on an ECG.

In some aspects, the disclosure provides a system for detecting aortic valve stenosis from an electrocardiogram signal(s), the system comprising: an input module receiving the electrocardiogram (ECG) signal from an information source; an analysis module trained to apply logic to identify an aortic valve stenosis pattern in one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, thirty or more, thirty-one or more, thirty-two or more, thirty-three or more, thirty-four or more, thirty-five or more, thirty-six or more, thirty-seven or more, thirty-eight or more, thirty-nine or more, forty or more, forty-one or more, forty-two or more, forty-three or more, forty-four or more, forty-five or more, forty-six or more, forty-seven or more, forty-eight or more, forty-nine or more, fifty or more, fifty-one or more, fifty-two or more, fifty-three or more, fifty-four or more, fifty-five or more, sixty-one or more, sixty-two or more, sixty-three or more, sixty-four or more, sixty-five or more, sixty-six or more, sixty-seven or more, sixty-eight or more, sixty-nine or more, seventy or more, seventy-one or more, seventy-two or more, seventy-three or more, seventy-four or more, seventy-five or more, seventy-six or more, seventy-seven or more, seventy-eight or more, seventy-nine or more, eighty or more, eighty-one or more, eighty-two or more, eighty-three or more, eighty-four or more, eighty-five or more, eighty-six or more of Patient's age at the time of ECG (in years), P wave duration in lead I, P wave duration in lead II, P wave duration in lead III, P wave duration in lead V₁, P wave duration in lead V₂, P wave duration in lead V₃, P wave duration in lead V₄, P wave duration in lead V₅, P wave duration in lead V₆, P wave duration in lead aVF, P wave duration in lead aVR, P wave duration in lead aVL, P wave amplitude in lead I, P wave amplitude in lead II, P wave amplitude in lead III, P wave amplitude in lead V₁, P wave amplitude in lead V₂, P wave amplitude in lead V₃, P wave amplitude in lead V₄, P wave amplitude in lead V₅, P wave amplitude in lead V₆, P wave amplitude in lead aVF, P wave amplitude in lead aVR, P wave amplitude in lead aVL, R wave amplitude in lead I, R wave amplitude in lead II, R wave amplitude in lead III, R wave amplitude in lead V₁, R wave amplitude in lead V₂, R wave amplitude in lead V₃, R wave amplitude in lead V₄, R wave amplitude in lead V₅, R wave amplitude in lead V₆, R wave amplitude in lead aVF, R wave amplitude in lead aVR, R wave amplitude in lead aVL, R wave duration in lead I, R wave duration in lead II, R wave duration in lead III, R wave duration in lead V₁, R wave duration in lead V₂, R wave duration in lead V₃, R wave duration in lead V₄, R wave duration in lead V₅, R wave duration in lead V₆, R wave duration in lead aVF, R wave duration in lead aVR, R wave duration in lead aVL, S wave amplitude in lead I, S wave amplitude in lead II, S wave amplitude in lead III, S wave amplitude in lead V₁, S wave amplitude in lead V₂, S wave amplitude in lead V₃, S wave amplitude in lead V₄, S wave amplitude in lead V₅, S wave amplitude in lead V₆, S wave amplitude in lead aVF, S wave amplitude in lead aVR, S wave amplitude in lead aVL, T wave amplitude in lead I, T wave amplitude in lead II, T wave amplitude in lead III, T wave amplitude in lead V₁, T wave amplitude in lead V₂, T wave amplitude in lead V₃, T wave amplitude in lead V₄, T wave amplitude in lead V₅, T wave amplitude in lead V₆, T wave amplitude in lead aVF, T wave amplitude in lead aVR, T wave amplitude in lead aVL, PRI value in lead I, PRI value in lead II, PRI value in lead III, PRI value in lead V₁, PRI value in lead V₂, PRI value in lead V₃, PRI value in lead V₄, PRI value in lead V₅, PRI value in lead V₆, PRI value in lead aVF, PRI value in lead aVL, PRI value in lead aVR, and/or a QT value; and an output module for outputting a classification (e.g., binary classification) indicative of a aortic valve stenosis pattern in the ECG signal or a non-cardiac aortic valve stenosis pattern in the ECG signal. In some instances, the analysis module is a feedforward neural network. In some instances, the system implements layers with a Heterogeneous Activations method, where each layer may have different activation functions depending on the task at that layer. ReLU activation functions is handle non-linear relationships in the data and final layer the sigmoid function which squashes the output maintaining probabilistic output calibration for risk scoring.

In some aspects, the disclosure provides a system for detecting aortic valve stenosis from an electrocardiogram signal(s), the system comprising: an input module receiving the electrocardiogram (ECG) signal from an information source; an analysis module trained to apply logic to identify a aortic valve stenosis pattern in no more than four, no more than five, no more than six, no more than seven, no more than eight, no more than nine, no more than ten, no more than eleven, no more than twelve, no more than thirteen, no more than fourteen, no more than fifteen, no more than sixteen, no more than seventeen, no more than eighteen, no more than nineteen, no more than twenty, no more than twenty-one, no more than twenty-two, no more than twenty-three, no more than twenty-four, no more than twenty-five, no more than twenty-six, no more than twenty-seven, no more than twenty-eight, no more than twenty-nine, no more than thirty, no more than thirty-one, no more than thirty-two, no more than thirty-three, no more than thirty-four, no more than thirty-five, no more than thirty-six, no more than thirty-seven, no more than thirty-eight, no more than thirty-nine, no more than forty, no more than forty-one, no more than forty-two, no more than forty-three, no more than forty-four, no more than forty-five, no more than forty-six, no more than forty-seven, no more than forty-eight, no more than forty-nine, no more than fifty, no more than fifty-one, no more than fifty-two, no more than fifty-three, no more than fifty-four, no more than fifty-five, no more than fifty-six, no more than fifty-seven, no more than fifty-eight, no more than fifty-nine, no more than sixty, no more than sixty-one, no more than sixty-two, no more than sixty-three, no more than sixty-four, no more than sixty-five, no more than sixty-six, no more than sixty-seven, no more than sixty-eight, no more than sixty-nine, no more than seventy, no more than seventy-one, no more than seventy-two, no more than seventy-three, no more than seventy-four, no more than seventy-five, no more than seventy-six, no more than seventy-seven, no more than seventy-eight, no more than seventy-nine, no more than eighty, no more than eighty-one, no more than eighty-two, no more than eighty-three, no more than eighty-four, no more than eighty-five, no more than eighty-six features selected from the group consisting of patient's age at the time of ECG (in years), P wave duration in lead I, P wave duration in lead II, P wave duration in lead III, P wave duration in lead V₁, P wave duration in lead V₂, P wave duration in lead V₃, P wave duration in lead V₄, P wave duration in lead V₅, P wave duration in lead V₆, P wave duration in lead aVF, P wave duration in lead aVR, P wave duration in lead aVL, P wave amplitude in lead I, P wave amplitude in lead II, P wave amplitude in lead III, P wave amplitude in lead V₁, P wave amplitude in lead V₂, P wave amplitude in lead V₃, P wave amplitude in lead V₄, P wave amplitude in lead V₅, P wave amplitude in lead V₆, P wave amplitude in lead aVF, P wave amplitude in lead aVR, P wave amplitude in lead aVL, R wave amplitude in lead I, R wave amplitude in lead II, R wave amplitude in lead III, R wave amplitude in lead V₁, R wave amplitude in lead V₂, R wave amplitude in lead V₃, R wave amplitude in lead V₄, R wave amplitude in lead V₅, R wave amplitude in lead V₆, R wave amplitude in lead aVF, R wave amplitude in lead aVR, R wave amplitude in lead aVL, R wave duration in lead I, R wave duration in lead II, R wave duration in lead III, R wave duration in lead V₁, R wave duration in lead V₂, R wave duration in lead V₃, R wave duration in lead V₄, R wave duration in lead V₅, R wave duration in lead V₆, R wave duration in lead aVF, R wave duration in lead aVR, R wave duration in lead aVL, S wave amplitude in lead I, S wave amplitude in lead II, S wave amplitude in lead III, S wave amplitude in lead V₁, S wave amplitude in lead V₂, S wave amplitude in lead V₃, S wave amplitude in lead V₄, S wave amplitude in lead V₅, S wave amplitude in lead V₆, S wave amplitude in lead aVF, S wave amplitude in lead aVR, S wave amplitude in lead aVL, T wave amplitude in lead I, T wave amplitude in lead II, T wave amplitude in lead III, T wave amplitude in lead V₁, T wave amplitude in lead V₂, T wave amplitude in lead V₃, T wave amplitude in lead V₄, T wave amplitude in lead V₅, T wave amplitude in lead V₆, T wave amplitude in lead aVF, T wave amplitude in lead aVR, T wave amplitude in lead aVL, PRI value in lead I, PRI value in lead II, PRI value in lead III, PRI value in lead V₁, PRI value in lead V₂, PRI value in lead V₃, PRI value in lead V₄, PRI value in lead V₅, PRI value in lead V₆, PRI value in lead aVF, PRI value in lead aVL, PRI value in lead aVR, and/or a QT value; and an output module for outputting a classification (e.g., binary classification) indicative of a aortic valve stenosis pattern in the ECG signal or a control group pattern in the ECG signal. In some instances, the analysis module is a feedforward neural network. An analysis module that is part of a system of the disclosure can be trained, e.g., on a dataset comprising at least 1,000 ECGs of subjects with AVS and at least 1,000 control subjects without heart disease. The analysis module can comprise a model that is a feedforward neural network trained on 62 parameters, including age, P wave amplitude, R wave amplitude, R wave duration, S wave amplitude, S wave duration, T wave amplitude, and average QT. In many implementations, the system implements multilayer deep-learning convolutional neural networks (CNNs) trained on vast digital ECG datasets, enabling the extraction of subtle relationship within these 62 parameters which are imperceptible to human experts. These networks autonomously learn complex spatiotemporal patterns across all 12 leads, facilitating the detection of nuanced electrical signatures associated with aortic valve stenosis and other cardiac pathologies.

In some aspects, a system described herein is optionally connected to a computer network. In further configurations, the system is optionally connected to the Internet such that it accesses the World Wide Web, e.g., the reference database of a plurality of normalized waveforms can be stored in the World Wide Web, including normalized waveforms that can provide a binary (“yes or no”) identification of aortic valve stenosis. In some embodiments, it is contemplated that the database comprises a plurality of ECG parameters indicative of a subject afflicted with aortic valve stenosis, e.g., for the purposes of training a model for detecting aortic valve stenosis and supporting detection of aortic valve stenosis. In still further configurations, the system is optionally connected to a cloud computing infrastructure. In other configurations, the digital processing device is optionally connected to an intranet. In other configurations, the digital processing device is optionally connected to a data storage device. In other configurations, the digital processing device could be deployed on premise or remotely deployed in the cloud. In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art. In many aspects, the disclosure contemplates any suitable system that can either be functionally connected with electrocardiogram ECG or EKG equipment, either directly or via a third-party, for on-site monitoring and aortic valve stenosis classification outputting (e.g., binary classification (0 to 1) or a classification associated with a particular type of aortic valve stenosis). Such systems can be seamlessly integrated with ECG devices at the point of care, hospital IT systems, requiring no new equipment or workflow changes. As demonstrated in the below examples, such systems were validated on patient datasets demonstrating high sensitivity, specificity, and AUC in large, multi-center datasets (e.g., Mayo Clinic, ARIC).

In some aspects, a system of the disclosure includes an operating system configured to perform executable instructions, e.g., receive via an input module electrocardiogram (ECG) signals from an information source (e.g., an ECG stored in the form of a medical record or a contemporaneous ECG). The operating system is, for example, software, including programs and data, which manages the overall system's hardware and provides services for execution of applications. Those of skill in the art will recognize that suitable server operating systems include, by way of non-limiting examples, FreeBSD, OpenBSD, NetBSD, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some aspects, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. In the specific Examples provided herein, the data was analyzed using IBM SPSS version 24.

In some aspects, an aortic valve stenosis detection system of the disclosure includes a storage and/or memory device. The storage and/or memory device can be one or more physical apparatuses used to store data or programs on a temporary or permanent basis. In some configurations, the device is volatile memory and requires power to maintain stored information. In some configurations, the device is non-volatile memory and retains stored information when the digital processing device is not powered. In further configurations, the non-volatile memory comprises flash memory. In some configurations, the non-volatile memory comprises dynamic random-access memory (DRAM). In some configurations, the non-volatile memory comprises ferroelectric random access memory (FRAM). In some configurations, the non-volatile memory comprises phase-change random access memory (PRAM). In other configurations, the device is a storage device including, by way of non-limiting examples, CD-ROMs, DVDs, flash memory devices, magnetic disk drives, magnetic tapes drives, optical disk drives, and cloud computing based storage. In further configurations, the storage and/or memory device is a combination of devices such as those disclosed herein.

In some configurations, an aortic valve stenosis system of the disclosure includes a display to send visual information to a third-party, such as health care facility, a physician's office, or a relative of the subject being monitored for aortic valve stenosis or undertaking an ECG. In some configurations, the display is a cathode ray tube (CRT). In some configurations, the display is a liquid crystal display (LCD). In further configurations, the display is a thin film transistor liquid crystal display (TFT-LCD). In some configurations, the display is an organic light emitting diode (OLED) display. In various further configurations, on OLED display is a passive-matrix OLED (PMOLED) or active-matrix OLED (AMOLED) display. In some configurations, the display is a plasma display. In other configurations, the display is a video projector. In still further configurations, the display is a combination of devices such as those disclosed herein. In certain configurations the performance of the deep neural network (DNN) in predicting aortic valve stenosis through a plurality of ECG parameters using one or more of the 12 lead ECG signals is outputted on the display, alongside its respective sensitivity or specificity for that range.

In some configurations, a aortic valve stenosis detection system includes an input device to receive information from a user. The information is then analyzed in the analysis module. In some configurations, the input device is an electrocardiogram machine, and the aortic valve stenosis detection system is formatted to receive a plurality of data from electrical activity of the heart as be measured on the surface of the skin, i.e., 12 lead ECG or a wrist-pulse ECG signal. This includes electrocardiogram data from one or more of: 1) RA electrode, placed on the right arm; 2) LA electrode, place on the left arm; 3) RL electrode, placed on the right leg; 4) LL electrode placed on the left leg; 5) V₁ electrode, placed in the fourth intercostal space (between ribs 4 and 5); 6) V₂ electrode, placed in the fourth intercostal space (between ribs 4 and 5); 7) V₃ electrode, placed between leads V₂ and V₄; 8) V₄ electrode, placed in the fifth intercostal space (between ribs 5 and 6); 9) V₅ electrode, placed horizontally even with V₄; and 10) V₆ electrode, placed horizontally even with V₄ and V₅ in the mid-axillary line. In still further configurations, the input device is a combination of devices such as those disclosed herein. In still further configurations, the input device detects wrist-pulse ECG signal data.

In some configurations, an aortic valve stenosis detection system includes a digital camera. In some configurations, a digital camera captures digital images, such as, e.g., a schematic representation of a ECG. In some configurations, a digital camera captures still images of the ECG for further analysis by the system's analysis module, and the system is able to segment the signal.

### Non-Transitory Computer Readable Storage Medium

In many aspects, the processes and systems that provide the aortic valve stenosis detection system disclosed herein include one or more non-transitory computer readable storage media encoded with a program including instructions executable by the operating system of an optionally networked digital processing device. For instance, in some aspects, the processes of the disclosure comprise creating data files associated with a plurality of ECG parameters from a set of data. In certain configurations, the system of the disclosure incorporates a database of normalized waveforms that can be used as a reference. In other configurations, a database of the disclosure may not require a reference database. The non-transitory computer storage medium can store data files associated with one or more 12-lead ECG measurements described herein.

Further the processes and systems that provide the aortic valve stenosis detection system disclosed herein can include one or more non-transitory computer readable storage media encoded with a program including instructions executable by the operating system of an optionally networked digital processing device configured to create data files associated with a plurality of waveforms (including raw ECG waveforms from one or more 12-lead ECG measurements, and parameters from all 12-lead ECG forms). In preferred configurations, the data is further analyzed by a system of the disclosure comprising a logic model that is trained to apply logic to identify an aortic valve stenosis pattern in one or more of: an R wave amplitude in lead V₅ of the ECG; an S wave amplitude in lead V₃ of the ECG; an R wave amplitude in lead aVL; an R wave amplitude in lead V₆; an R wave amplitude in lead V₄; an R wave amplitude in lead I; an S wave amplitude in lead V₁; and an S wave amplitude in lead V₂. It is contemplated that slightly different models may identify slightly different parameters. In combination with the analysis of the ECG parameters, the output of the process described herein can provide a binary classification of aortic valve stenosis based on the diagnosis code. The non-transitory computer storage medium can store data files associated with all of the aortic valve stenosis designations described herein.

In further configurations, a computer readable storage medium is a tangible component of a system of the disclosure. In still further configurations, a computer readable storage medium is optionally removable from a digital processing device. In some configurations, a computer readable storage medium includes, by way of non-limiting examples, CD-ROMs, DVDs, flash memory devices, solid state memory, magnetic disk drives, magnetic tape drives, optical disk drives, storage area network (SAN), cloud computing systems and services, and the like. In some cases, the program and instructions are permanently, substantially permanently, semi-permanently, or non-transitorily encoded on the media. Such computer readable storage medium is also suitable for storing the set of data contemplated by the disclosure.

### Computer Program

The processes and systems that provide the aortic valve stenosis identification disclosed herein typically include at least one computer program. A computer program includes a sequence of instructions, executable in the digital processing device's CPU, written to perform a specified task. Example 1 provides exemplary steps used by Applicants to, e.g., develop the system and methodology suitable for identification of aortic valve stenosis from ECG parameters. The examples also demonstrate the sensitivity of an exemplary system. The S-G filter, for instance, is a digital filter that effectively reduces noise and unwanted variations in the signal while preserving the underlying prominent features.

In light of the disclosure provided herein, those of skill in the art will recognize that a computer program may be written in various versions of various languages. In some configurations, a computer program comprises one sequence of instructions. For instance, a program may be written to achieve the same sequence of instructions or substantially the same sequence of instructions instantly described to develop a aortic valve stenosis model. For instance, in developing the model, the disclosure contemplates systems having computer programs configured to 1) quantify the disparity between the model's predictions and the true binary labels, guiding the optimization process to minimize this discrepancy (e.g., loss=‘binary_crossentropy’); 2) dynamically updating the model's weights during training (e.g., optimizer=‘adam’: The “optimizer”); 3) measuring the ratio of correctly predicted instances to the total instances (e.g., metrics=[‘accuracy’]); 4) dynamically monitor plateaus during training (e.g., reduce_Ir=ReduceLROnPlateau( )); and 5) to prevent overfitting (e.g., early_stopping=EarlyStopping(patience=50, min_delta=0.0001). A program may be written to achieve the same sequence of instructions or substantially the same sequence of instructions instantly described to apply a aortic valve stenosis detection system to the classification of one or more 12 ECG signals associated with aortic valve stenosis.

In some configurations, a computer program instruction is provided from one location (e.g., a computer program that is functionally connected to an ECG apparatus or another medical apparatus). In other configurations, a computer program is provided from a plurality of locations (e.g., ECG signal is provide to a third-party and the analysis occurs in yet another site). In various configurations, a computer program includes one or more software modules. In various configurations, a computer program includes, in part or in whole, one or more web applications, one or more mobile applications, one or more standalone applications, one or more web browser plug-ins, extensions, add-ins, or add-ons, or combinations thereof.

### Web Application

In some configurations, the processes and systems that provide the aortic valve stenosis detection system disclosed herein include a web application. In light of the disclosure provided herein, those of skill in the art will recognize that a web application, in various configurations, utilizes one or more software frameworks and one or more database systems. In some configurations, a web application is created upon a software framework such as Microsoft®.NET or Ruby on Rails (RoR). In some configurations, a web application utilizes one or more database systems including, by way of non-limiting examples, relational, non-relational, object oriented, associative, and XML database systems. In further configurations, suitable relational database systems include, by way of non-limiting examples, Microsoft® SQL Server, mySQL™, and Oracle®. Those of skill in the art will also recognize that a web application, in various configurations, is written in one or more versions of one or more languages. A web application may be written in one or more markup languages, presentation definition languages, client-side scripting languages, server-side coding languages, database query languages, or combinations thereof. In some configurations, a web application is written to some extent in a markup language such as Hypertext Markup Language (HTML), Extensible Hypertext Markup Language (XHTML), or eXtensible Markup Language (XML). In some configurations, a web application is written to some extent in a presentation definition language such as Cascading Style Sheets (CSS). In some configurations, a web application is written to some extent in a client-side scripting language such as Asynchronous Javascript and XML (AJAX), Flash® Actionscript, Javascript, or Silverlight®. In some configurations, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some configurations, a web application is written to some extent in a database query language such as Structured Query Language (SQL). In some configurations, a web application integrates enterprise server products such as IBM® Lotus Domino®. A web application for providing a career development network for artists that allows artists to upload information and media files, in some configurations, includes a media player element. In various further configurations, a media player element utilizes one or more of many suitable multimedia technologies including, by way of non-limiting examples, Adobe® Flash®, HTML 5, Apple® QuickTime®, Microsoft® Silverlight®, Java™ and Unity®.

### Mobile Application

In some configurations, the systems that provide the aortic valve stenosis detection system disclosed herein include a mobile application provided to a mobile digital processing device. In some configurations, the mobile application is provided to a mobile digital processing device at the time it is manufactured. In other configurations, the mobile application is provided to a mobile digital processing device via the computer network described herein. It is specifically contemplated that the aortic valve stenosis detection system is configured for display on a mobile device. In specific instances, the aortic valve stenosis detection system outputs a classification result for display on an interface, e.g., graphical user interface. In certain configurations, the aortic valve stenosis classification output comprises one or a combination of two or more of text, color, imagery, or sound to alert the subject or the technician, physician, or the like administering the ECG of a subject.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some configurations, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some configurations, a computer program includes one or more executable complied applications.

### Software Modules

The processes and systems that provide the aortic valve stenosis detection system disclosed herein include, in various configurations, software, server, and database modules. A specific database contemplated by the disclosure is described in the Examples. In view of the disclosure provided herein, software modules are created by techniques known to those of skill in the art using machines, software, and languages known to the art. The software modules disclosed herein are implemented in a multitude of ways. In various configurations, a software module comprises a file, a section of code, a programming object, a programming structure, or combinations thereof. In further various configurations, a software module comprises a plurality of files, a plurality of sections of code, a plurality of programming objects, a plurality of programming structures, or combinations thereof. In various configurations, the one or more software modules comprise, by way of non-limiting examples, a web application, a mobile application, and a standalone application. In some configurations, software modules are in one computer program or application. In other configurations, software modules are in more than one computer program or application. In some configurations, software modules are hosted on one machine. In other configurations, software modules are hosted on more than one machine. In further configurations, software modules are hosted on cloud computing platforms. In some configurations, software modules are hosted on one or more machines in one location. In other configurations, software modules are hosted on one or more machines in more than one location.

A system with the characteristics outlined herewith has demonstrated clinical utility in:
A) early Detection: Identifies moderate-to-severe AS up to 2 years before typical intervention.
B) Risk Stratification/Prognostics: Distinguishes high-risk phenotypes based on longitudinal score trajectories.
C) Predictive: Predicts adverse outcomes post-TAVR, including mortality and pacemaker need.
D) Resource Optimization: Reduces unnecessary echocardiograms and enables targeted follow-up.

### Embodiments

EMBODIMENT 1. An embodiment provides a process for screening an ECG for a pattern that is predictive of aortic valve stenosis, the process comprising: screening a plurality of parameters from one or more electrocardiogram (ECG) leads for a pattern that is predictive of aortic valve stenosis, whereby the screening is performed by a computer program model trained to identify the predictive pattern from the plurality of parameters, whereby: at least a first parameter in the plurality of parameters is an age of a subject associated with the ECG; and at least a second parameter is selected from one or more of P wave amplitude, R wave amplitude, R wave duration, S wave amplitude, S wave duration, T wave amplitude, and QT interval, and outputting a value that is predictive of aortic valve stenosis based on the pattern that is predictive of aortic valve stenosis.

EMBODIMENT 2. The process of EMBODIMENT 1, wherein the at least one second parameter is the QT interval.

EMBODIMENT 3. The process of any one of EMBODIMENTS 1-2, whereby at least one additional parameter is a T amplitude from lead V4.

EMBODIMENT 4. The process of any one of EMBODIMENTS 1-3, whereby at least one additional parameter is a T amplitude from lead AVL.

EMBODIMENT 5. The process of any one of EMBODIMENTS 1-4, whereby at least one additional parameter is a R amplitude from lead II.

EMBODIMENT 6. The process of any one of EMBODIMENTS 1-5, whereby at least one additional parameter is a P wave amplitude of lead I.

EMBODIMENT 7. The process of any one of EMBODIMENTS 1-6, whereby at least one additional parameter is a P wave amplitude of lead II.

EMBODIMENT 8. The process of any one of EMBODIMENTS 1-7, whereby at least one additional parameter is a P wave amplitude of lead III.

EMBODIMENT 9. The process of any one of EMBODIMENTS 1-8, whereby at least one additional parameter is a P wave amplitude of lead V1.

EMBODIMENT 10. The process of any one of EMBODIMENTS 1-9, whereby at least one additional parameter is a P wave amplitude of lead V2.

EMBODIMENT 11. The process of any one of EMBODIMENTS 1-10, whereby at least one additional parameter is a P wave amplitude of lead V3.

EMBODIMENT 12. The process of any one of EMBODIMENTS 1-11, whereby at least one additional parameter is a P wave amplitude of lead V4.

EMBODIMENT 13. The process of any one of EMBODIMENTS 1-12, whereby at least one additional parameter is a P wave amplitude of lead V5.

EMBODIMENT 14. The process of any one of EMBODIMENTS 1-13, whereby at least one additional parameter is a P wave amplitude of lead V6.

EMBODIMENT 15. The process of any one of EMBODIMENTS 1-14, whereby at least one additional parameter is a P wave amplitude of lead aVF.

EMBODIMENT 16. The process of any one of EMBODIMENTS 1-15, whereby at least one additional parameter is a P wave amplitude of lead aVR.

EMBODIMENT 17. The process of any one of EMBODIMENTS 1-16, whereby at least one additional parameter is a P wave amplitude of lead aVL.

EMBODIMENT 18. The process of any one of EMBODIMENTS 1-17, whereby at least one additional parameter is a R wave amplitude of lead I.

EMBODIMENT 19. The process of any one of EMBODIMENTS 1-18, whereby at least one additional parameter is a R wave amplitude of lead III.

EMBODIMENT 20. The process of any one of EMBODIMENTS 1-19, whereby at least one additional parameter is a R wave amplitude of lead V₁.

EMBODIMENT 21. The process of any one of EMBODIMENTS 1-20, whereby at least one additional parameter is a R wave amplitude of lead V2.

EMBODIMENT 22. The process of any one of EMBODIMENTS 1-21, whereby at least one additional parameter is a R wave amplitude of lead V3.

EMBODIMENT 23. The process of any one of EMBODIMENTS 1-22, whereby at least one additional parameter is a R wave amplitude of lead V4.

EMBODIMENT 24. The process of any one of EMBODIMENTS 1-23, whereby at least one additional parameter is a R wave amplitude of lead V5.

EMBODIMENT 25. The process of any one of EMBODIMENTS 1-24, whereby at least one additional parameter is a R wave amplitude of lead V6.

EMBODIMENT 26. The process of any one of EMBODIMENTS 1-25, whereby at least one additional parameter is a R wave amplitude of lead aVF.

EMBODIMENT 27. The process of any one of EMBODIMENTS 1-26, whereby at least one additional parameter is a R wave amplitude of lead aVR.

EMBODIMENT 28. The process of any one of EMBODIMENTS 1-27, whereby at least one additional parameter is a R wave amplitude of lead aVL.

EMBODIMENT 29. The process of any one of EMBODIMENTS 1-28, whereby at least one additional parameter is a R wave duration of lead I.

EMBODIMENT 30. The process of any one of EMBODIMENTS 1-29, whereby at least one additional parameter is a R wave duration of lead II.

EMBODIMENT 31. The process of any one of EMBODIMENTS 1-30, whereby at least one additional parameter is a R wave duration of lead III.

EMBODIMENT 32. The process of any one of EMBODIMENTS 1-31, whereby at least one additional parameter is a R wave duration of lead V1.

EMBODIMENT 33. The process of any one of EMBODIMENTS 1-32, whereby at least one additional parameter is a R wave duration of lead V2.

EMBODIMENT 34. The process of any one of EMBODIMENTS 1-33, whereby at least one additional parameter is a R wave duration of lead V3.

EMBODIMENT 35. The process of any one of EMBODIMENTS 1-34, whereby at least one additional parameter is a R wave duration of lead V4.

EMBODIMENT 36. The process of any one of EMBODIMENTS 1-35, whereby at least one additional parameter is a R wave duration of lead V5.

EMBODIMENT 37. The process of any one of EMBODIMENTS 1-36, whereby at least one additional parameter is a R wave duration of lead V6.

EMBODIMENT 38. The process of any one of EMBODIMENTS 1-37, whereby at least one additional parameter is a R wave duration of lead aVF.

EMBODIMENT 39. The process of any one of EMBODIMENTS 1-38, whereby at least one additional parameter is a R wave duration of lead aVR.

EMBODIMENT 40. The process of any one of EMBODIMENTS 1-39, whereby at least one additional parameter is a R wave duration of lead aVL.

EMBODIMENT 41. The process of any one of EMBODIMENTS 1-40, whereby at least one additional parameter is a S wave amplitude of lead I.

EMBODIMENT 42. The process of any one of EMBODIMENTS 1-41, whereby at least one additional parameter is a S wave amplitude of lead II.

EMBODIMENT 43. The process of any one of EMBODIMENTS 1-42, whereby at least one additional parameter is a S wave amplitude of lead III.

EMBODIMENT 44. The process of any one of EMBODIMENTS 1-43, whereby at least one additional parameter is a S wave amplitude of lead V1.

EMBODIMENT 45. The process of any one of EMBODIMENTS 1-44, whereby at least one additional parameter is a S wave amplitude of lead V2.

EMBODIMENT 46. The process of any one of EMBODIMENTS 1-45, whereby at least one additional parameter is a S wave amplitude of lead V3.

EMBODIMENT 47. The process of any one of EMBODIMENTS 1-46, whereby at least one additional parameter is a S wave amplitude of lead V4.

EMBODIMENT 48. The process of any one of EMBODIMENTS 1-47, whereby at least one additional parameter is a S wave amplitude of lead V5.

EMBODIMENT 49. The process of any one of EMBODIMENTS 1-48, whereby at least one additional parameter is a S wave amplitude of lead V6.

EMBODIMENT 50. The process of any one of EMBODIMENTS 1-49, whereby at least one additional parameter is a S wave amplitude of lead aVF.

EMBODIMENT 51. The process of any one of EMBODIMENTS 1-50, whereby at least one additional parameter is a S wave amplitude of lead a VR.

EMBODIMENT 52. The process of any one of EMBODIMENTS 1-51, whereby at least one additional parameter is a S wave amplitude of lead aVL.

EMBODIMENT 53. The process of any one of EMBODIMENTS 1-52, whereby at least one additional parameter is a T wave amplitude of lead I.

EMBODIMENT 54. The process of any one of EMBODIMENTS 1-53, whereby at least one additional parameter is a T wave amplitude of lead II.

EMBODIMENT 55. The process of any one of EMBODIMENTS 1-54, whereby at least one additional parameter is a T wave amplitude of lead III.

EMBODIMENT 56. The process of any one of EMBODIMENTS 1-55, whereby at least one additional parameter is a T wave amplitude of lead V1.

EMBODIMENT 57. The process of any one of EMBODIMENTS 1-56, whereby at least one additional parameter is a T wave amplitude of lead V2.

EMBODIMENT 58. The process of any one of EMBODIMENTS 1-57, whereby at least one additional parameter is a T wave amplitude of lead V3.

EMBODIMENT 59. The process of any one of EMBODIMENTS 1-58, whereby at least one additional parameter is a T wave amplitude of lead V5.

EMBODIMENT 60. The process of any one of EMBODIMENTS 1-59, whereby at least one additional parameter is a T wave amplitude of lead V6.

EMBODIMENT 61. The process of any one of EMBODIMENTS 1-60, whereby at least one additional parameter is a T wave amplitude of lead aVF.

EMBODIMENT 62. The process of any one of EMBODIMENTS 1-61, whereby at least one additional parameter is a T wave amplitude of lead aVR.

EMBODIMENT 63. The process of EMBODIMENT 2, wherein the pattern that is predictive of aortic valve stenosis is a reduced QT interval as a function of the age of the subject associated with the ECG.

EMBODIMENT 64. The process of any one of EMBODIMENTS 1-63, wherein a negative predictive value of the pattern is greater than 90%.

EMBODIMENT 65. The process of any one of EMBODIMENTS 1-64, wherein the aortic valve stenosis is selected from the group consisting of rheumatic disorder of both mitral and aortic valve, nonrheumatic AVS, nonrheumatic AVS with insufficiency, congenital stenosis of AVS, bicuspid aortic valve, congenital insufficiency of aortic valve, aortic insufficiency/stenosis, nonrheumatic aortic valve disorder, unspecified other nonrheumatic AVS, stenosis aortic moderate, stenosis aortic congenital, regurgitation aortic, rheumatic aortic stenosis with insufficiency, congenital subaortic stenosis, supravalvular aortic stenosis, rheumatic aortic stenosis with insufficiency, stenosis aortic rheumatic, aortic valve disease, aortic regurgitation, and rheumatic aortic stenosis.

EMBODIMENT 66. The process of any one of EMBODIMENTS 1-65, wherein the sensitivity of the value that is predictive of aortic valve stenosis is greater than 70%.

EMBODIMENT 67. The process of any one of EMBODIMENTS 1-66, wherein the specificity of the value that is predictive of aortic valve stenosis is greater than 70%.

EMBODIMENT 68. The process of any one of EMBODIMENTS 1-67, wherein the computer program is trained on a dataset that comprises at least 1,000 ECG's of subjects with at least one form of AVS and at least 1,000 control subjects who did not have a diagnosis of heart disease.

EMBODIMENT 69. The process of any one of EMBODIMENTS 1-68, further comprising a step of treating a subject associated with the ECG for the aortic valve stenosis.

EMBODIMENT 70. The process of any one of EMBODIMENTS 1-69, wherein the computer program model is a feedforward neural network model.

EMBODIMENT 71. The process of any one of EMBODIMENTS 1-70, wherein the computer program model is trained on 62 parameters comprising the age of a subject associated with the ECG, the P wave amplitude, the R wave amplitude, the R wave duration, the S wave amplitude, the S wave duration, the T wave amplitude, and the average QT.

EMBODIMENT 72. The process of any one of EMBODIMENTS 1-71, wherein a user directs the training of the model to adjust the learning rate dynamically based on the plateauing of a monitored metric.

EMBODIMENT 73. The process of any one of EMBODIMENTS 1-72, wherein a user directs the training of the model to prevent overfitting by monitoring validation metrics.

EMBODIMENT 74. The process of any one of EMBODIMENTS 1-73, whereby the

ECG is from a subject that is at least 18 years or older.

EMBODIMENT 75. The process of EMBODIMENT 74, whereby the ECG is from a subject that is at least 22 years or older.

EMBODIMENT 76. The process of EMBODIMENT 74, whereby the subject does not have a pacemaker.

EMBODIMENT 77. The process of EMBODIMENT 74, whereby the subject has not suffered a prior myocardial infarction, a left ventricular hypertrophy, or cardiac surgery.

EMBODIMENT 78. The process of any one of EMBODIMENTS 1-77, wherein the output further comprises a risk score indicating the probability of developing moderate or severe aortic stenosis within a predefined time period following a negative echocardiogram.

EMBODIMENT 79. The process of any one of EMBODIMENTS 1-77, wherein the output further comprises a risk score indicating the probability of developing heart failure within a predefined time period following a negative echocardiogram.

EMBODIMENT 80. The process of any one of EMBODIMENTS 1-79, wherein the computer program model is further configured to analyze longitudinal ECG data from a subject and to classify the subject into a risk trajectory cluster, wherein the risk trajectory cluster is associated with a distinct prognosis for mortality or adverse cardiovascular outcomes.

EMBODIMENT 81. The process of any one of EMBODIMENTS 1-79, wherein the computer program model is further configured to analyze periprocedural changes in the risk score before and after aortic valve intervention, and to output a prognostic indicator of 1-year mortality, risk of permanent pacemaker implantation, or length of hospital stay.

EMBODIMENT 82. The process of any one of EMBODIMENTS 1-81, wherein a positive risk score in the absence of echocardiographic evidence of aortic stenosis is associated with a statistically significant increased risk of developing moderate or severe aortic stenosis or heart failure within five years.

EMBODIMENT 83. The process of any one of EMBODIMENTS 1-82, wherein the output is used to trigger automated alerts or referrals for further diagnostic evaluation or early intervention in a hospital electronic health record system.

EMBODIMENT 84. The process of any one of EMBODIMENTS 1-83, wherein the computer program model is trained and validated on datasets comprising at least 100,000 patients and is configured to maintain predictive accuracy across diverse demographic groups.

EMBODIMENT 85. The process of any one of EMBODIMENTS 1-84, wherein the output further comprises a recommendation for timing of aortic valve intervention based on the subject's risk trajectory cluster and predicted clinical outcomes

EMBODIMENT 86. The process of any one of EMBODIMENTS 1-85, wherein the computer program model is further configured to provide a risk score for adverse outcomes following transcatheter aortic valve replacement, including mortality, need for permanent pacemaker, and length of hospital stay.

EMBODIMENT 87. The process any one of EMBODIMENTS 1-86, wherein the computer program model is further configured to provide a risk score for future onset of aortic stenosis in subjects with false-positive screening results, and wherein the risk score is used to guide longitudinal surveillance.

EMBODIMENT 88. An embodiment provides a system for screening an ECG for a pattern that is predictive of aortic valve stenosis, the system comprising: an input module for receiving a plurality of parameters from one or more electrocardiogram (ECG) leads; an analysis module comprising a computer program model trained to identify a pattern predictive of aortic valve stenosis from the plurality of parameters from at least two inputs: an age of a subject associated with the ECG; and one or more of P wave amplitude, R wave amplitude, R wave duration, S wave amplitude, S wave duration, T wave amplitude, and QT interval, and outputting a value that is predictive of aortic valve stenosis based on the pattern that is predictive of aortic valve stenosis.

EMBODIMENT 89. The system of EMBODIMENT 88, wherein the analysis module identifies the pattern predictive of aortic valve stenosis from no more than two said inputs: the age of the subject associated with the ECG and the QT interval.

EMBODIMENT 90. The system of any one of EMBODIMENTS 88-89, wherein a negative predictive value of the pattern predictive of aortic valve stenosis is greater than 90%.

EMBODIMENT 91. The system of any one of EMBODIMENTS 88-90, wherein a sensitivity of the identification of the pattern that is predictive of aortic valve stenosis is greater than 70%.

EMBODIMENT 92. The system of any one of EMBODIMENTS 88-91, wherein a specificity of the identification of the pattern that is predictive of aortic valve stenosis is greater than 70%.

EMBODIMENT 93. The system of any one of EMBODIMENTS 88-92, wherein the analysis module identifies a pattern predictive of aortic valve stenosis from at least three inputs selected from the group consisting of the age of the subject associated with the ECG, the QT interval, a T amplitude from lead V4, a T amplitude from lead AVL, and R amplitude from lead II.

EMBODIMENT 94. The system of EMBODIMENT 93, wherein the pattern that is predictive of aortic valve stenosis is a reduced QT interval as a function of the age of the subject associated with the ECG.

EMBODIMENT 95. The system of EMBODIMENT 93, wherein the pattern that is predictive of aortic valve stenosis is an increased T amplitude from lead V4 as a function of the age of the subject associated with the ECG.

EMBODIMENT 96. The system of any one of EMBODIMENTS 88-95, wherein the aortic valve stenosis is selected from the group consisting of rheumatic disorder of both mitral and aortic valve, nonrheumatic AVS, nonrheumatic AVS with insufficiency, congenital stenosis of AVS, bicuspid aortic valve, congenital insufficiency of aortic valve, aortic insufficiency/stenosis, nonrheumatic aortic valve disorder, unspecified other nonrheumatic AVS, stenosis aortic moderate, stenosis aortic congenital, regurgitation aortic, rheumatic aortic stenosis with insufficiency, congenital subaortic stenosis, supravalvular aortic stenosis, rheumatic aortic stenosis with insufficiency, stenosis aortic rheumatic, aortic valve disease, aortic regurgitation, and rheumatic aortic stenosis.

EMBODIMENT 97. The system of any one of EMBODIMENTS 88-96, wherein the computer program is trained on a dataset that comprises at least 1,000 ECG's of subjects with at least one form of AVS and at least 1,000 control subjects who did not have a diagnosis of heart disease.

EMBODIMENT 98. The system of any one of EMBODIMENTS 88-97, wherein the computer program model is a feedforward neural network model.

EMBODIMENT 99. The system of any one of EMBODIMENTS 88-98, wherein the computer program model is trained on 62 parameters comprising the age of a subject associated with the ECG, the P wave amplitude, the R wave amplitude, the R wave duration, the S wave amplitude, the S wave duration, the T wave amplitude, and the average QT.

EMBODIMENT 100. The system of any one of EMBODIMENTS 88-99, wherein a user directs the training of the model to adjust the learning rate dynamically based on the plateauing of a monitored metric.

EMBODIMENT 101. The system of any one of EMBODIMENTS 88-100, wherein a user directs the training of the model to prevent overfitting by monitoring validation metrics.

EMBODIMENT 102. The system of any one of EMBODIMENTS 88-101, whereby the subject is at least 18 years or older.

EMBODIMENT 103. The system of EMBODIMENT 102, whereby the subject is at least 22 years or older.

EMBODIMENT 104. The system of any one of EMBODIMENTS 88-103, whereby the subject does not have a pacemaker.

EMBODIMENT 105. The system of any one of EMBODIMENTS 88-104, whereby the subject has not suffered a prior myocardial infarction, a left ventricular hypertrophy, or cardiac surgery.

### EXAMPLES

### Example 1—Model Development for Aortic Valve Stenosis (AVS) Detection

This example details the development of a robust model and an analysis module for aortic valve stenosis detection:

### Database Development

The instant model was developed as part of the Mayo Clinic Platform Accelerate Cohort 3 Dataset; this dataset encompasses of 2.8 million patients and 9.5 million ECGS. Each patient's data profile is characterized by 8 distinct waveforms, aligning with the 8 different leads: I, II, V1, V2, V3, V4, V5, and V6. These waveforms were originally encoded in the base64 format. Each waveform extends across a time span of 10 seconds operating at either 250 or 500 samples per second. These ECG records are recorded using a GE machine.

For all the ECG data, a dedicated database was created that contains measurements corresponding to each ECG record. This setup supports the association of specific measurements with individual ECG records. This data was accessed using SQL queries.

A cohort was created for all the adult patients at the Mayo Clinic between 2017 and 2022 who were above the age of 22. The patient cohort was carefully selected based on specific criteria to ensure a focused and comprehensive study. To be included in this cohort, patients had to meet two key conditions. Firstly, they must have undergone at least one echocardiogram during their visit to Mayo Hospital. This echocardiogram serves as a critical diagnostic component to assess cardiac health. Secondly, their medical records should contain a diagnosis code indicative of any form of Aortic valve stenosis. Meeting both conditions ensures that the patients included in this cohort have a strong association with AVS-related health concerns.

Following the diagnosis of AVS, examination of patient records was conducted to gather comprehensive data for analysis. The group comprised of patients who had undergone both an echocardiogram (Echo) and an electrocardiogram (ECG) within a 180-day window around the time of diagnosis. This timeframe was selected to capture crucial clinical information surrounding the identification of AVS, allowing for a detailed understanding of the immediate clinical context.

Patients with pacemakers were excluded from the study to ensure a more homogeneous group for analysis, as pacemakers can significantly affect cardiac parameters and potentially confound the interpretation of data related to AVS.

In addition to AVS, these patients also presented with other comorbidities:
a) approximately 40% of the patients had experienced a myocardial infarction (MI), indicating a history of heart attacks;
b) around 43% of the patients had chronic kidney disease (CKD), suggesting impaired kidney function as an additional health concern;
c) nearly half of the patients (49%) had been diagnosed with heart failure, indicating a significant burden on the heart's pumping ability;
d) a small percentage (4.7%) of patients had inflammatory heart diseases, indicating underlying inflammation affecting the heart;
e) some patients (2.4%) had metabolic heart diseases, which might involve disorders related to how the body processes energy;
f) a substantial portion (79%) of the patients had a history of cancer, indicating the presence of malignancies alongside cardiovascular issues;
g) about 16% of patients had cardiomyopathy, which involves abnormalities in the heart muscle structure or function.

This diverse spectrum of comorbidities underscores the complexity of managing patients with AVS and highlights the need for a multidisciplinary approach to patient care. Understanding how these conditions interact with AVS is essential for developing effective treatment strategies and improving patient outcomes.

The Table 1 AVS ICD codes below provides a comprehensive list of the different types of AVS that were included in the training and validation process.

TABLE 1

| CODE | DESCRIPTION |
|---|---|
| 396.0, 396.2, 396.1, 396.3 | Mitral and AVS |
| I08.0 | Rheumatic Disorder of Both Mitral and Aortic Valve |
| I35.0, 135.2,135.1 | Nonrheumatic AVS, Nonrheumatic AVS with Insufficiency |
| Q23.0 | Congenital Stenosis Of AVS |
| Q23.1, 746.4, 746.3 | Bicuspid Aortic Valve, Congenital Insufficiency of Aortic Valve |
| 424.1 | Aortic Insufficiency/Stenosis |
| I35.9 | Nonrheumatic Aortic Valve Disorder, Unspecified |
| I35.8 | Other Nonrheumatic AVS |
| 3959530 | Stenosis Aortic Moderate |
| 747.22 | Stenosis Aortic Congenital |
| 3959612 | Regurgitation Aortic |
| 395.2 | Rheumatic Aortic Stenosis with Insufficiency |
| Q24.4 | Congenital Subaortic Stenosis |
| Q25.3 | Supravalvular Aortic Stenosis |
| I06.2 | Rheumatic Aortic Stenosis with Insufficiency |
| I06.0, 395.0 | Stenosis Aortic Rheumatic |
| 1235209017 | Aortic Valve Disease |
| 100062010 | Aortic Regurgitation |
| 11960019 | Rheumatic Aortic Stenosis |

The list of disease classes mentioned here encompasses the following cardiac conditions.

TABLE 2
Disease Class Definition

| Class | Diseases/Conditions |
|---|---|
| Myocardial Infarction | ST-segment elevation myocardial infarction (STEMI), |
| | Non-ST-segment elevation myocardial infarction |
| | (NSTEMI), |
| | Q-wave myocardial infarction, |
| | Non-Q-wave myocardial infarction, |
| | Cardiac Aneurysm |
| Valvular Heart Diseases | Aortic Stenosis, |
| | Aortic Regurgitation, |
| | Mitral Stenosis, |
| | Mitral Regurgitation, |
| | Tricuspid Valve Disease, |
| | Pulmonary Valve Disease |
| | Tricuspid Valve Insufficiency, |
| | Aortic Insufficiency, |
| | Mitral Insufficiency. |
| Cardiomyopathies | Dilated Cardiomyopathy (DCM), |
| | Hypertrophic Cardiomyopathy (HCM), |
| | Restrictive Cardiomyopathy (RCM), |
| | Arrhythmogenic Right Ventricular Cardiomyopathy |
| | (ARVC), |
| | Cardiotoxicity (E.G., Chemotherapy-Induced |
| | Cardiomyopathy), |
| | Takotsubo Cardiomyopathy (Stress-Induced |
| | Cardiomyopathy), |
| | Ventricular Noncompaction Cardiomyopathy |
| Inflammatory Heart Diseases | Pericarditis (Including Myocarditis), |
| | Rheumatic Heart Disease, |
| | Myocarditis |
| Metabolic Heart Disorders | Amyloidosis, |
| | Fabry Disease, |
| | Hemochromatosis, |
| | Glycogen Storage Diseases, |
| | Lipid Storage Disorders |
| Congenital Heart Defects | Atrial Septal Defect (ASD) |
| | Ventricular Septal Defect (VSD) |
| | Tetralogy Of Fallot |
| | Patent Ductus Arteriosus (PDA) |
| | Coarctation Of The Aorta |
| | Ebstein's Anomaly |
| Heart Failure | Left Heart Failure, |
| | Diastolic Heart Failure, |
| | Heart Failure, |
| | Right Heart Failure, |
| | Systolic Heart Failure |
| CKD | Chronic Kidney Disease |
| Cancer | Cancer |

This control group was meticulously curated from the same original pool of adult patients who had sought medical attention at the Mayo Clinic between 2017 and 2022 and were aged 22 years or older.

The control group also comprised patients who had undergone both an echocardiogram (Echo) and an electrocardiogram (ECG) within a 180-day window around the time of diagnosis, mirroring the data collection protocol of the study group.

To maintain consistency and avoid potential confounding factors, several exclusions were applied to the control group. Patients with pacemakers were excluded from consideration, as were those diagnosed with valvular heart diseases, amyloidosis, cardiomyopathy, chronic kidney disease (CKD), heart failure, and inflammatory heart diseases. Moreover, the composition of the control group reflected a diverse array of health conditions commonly found within the general population. Approximately 15% of individuals in the control group had a history of myocardial infarction (MI). Additionally, a subset of patients (3%) within the control group exhibited congenital heart defects.

Furthermore, a significant proportion (68%) of individuals in the control group had been diagnosed with cancer, emphasizing the prevalence and impact of oncological diseases among adults attending the Mayo Clinic.

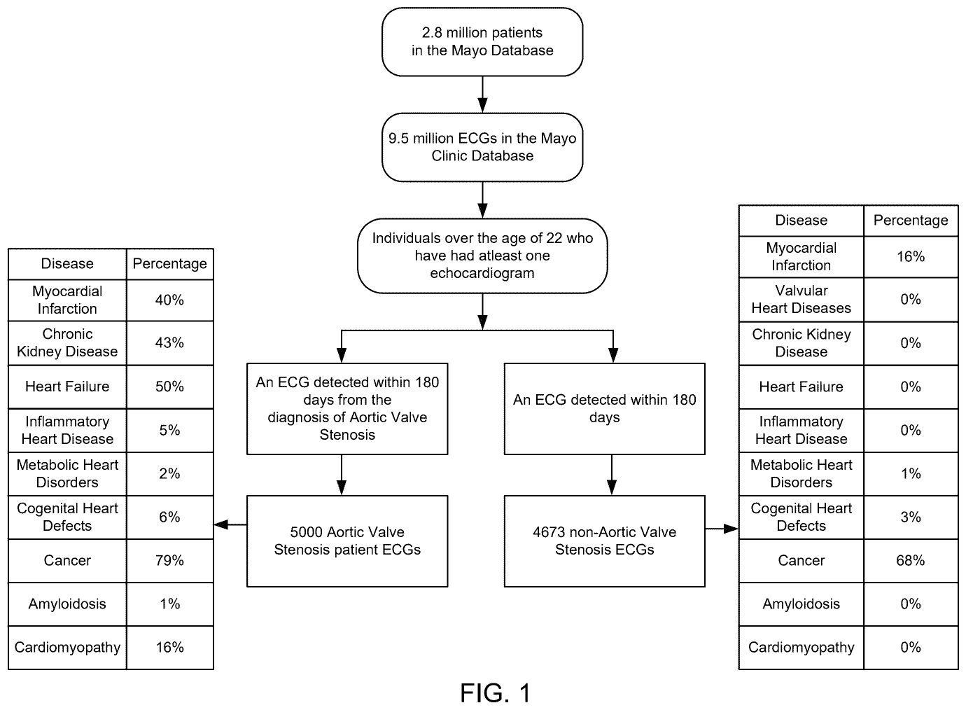

The model was trained on 9673 patients and internally tested using the development data set. The Baseline Characteristics of Development set is provided in Table 3 below. Table 3 (below) provides an overview of the patient demographics within the Development dataset. A sample of 9673 was collected for training. In this dataset 51% of the patients were identified as male and 48% as female. The mean age for the training set was 79 years for patients with AVS and 59 years for patients without AVS. FIG. 1 provides a flow chart of how the data was curated for development.

TABLE 3

| | Overall | | Test (AVS) | | Control | |
|---|---|---|---|---|---|---|
| Patients count | 9673 | | 5000 | | 4673 | |
| Gender | | | | | | |
| Male | 4974 | (51.4%) | 2847 | (56.9%) | 2127 | (45.5%) |
| Female | 4699 | (48.5%) | 2153 | (43.0%) | 2546 | (54.4%) |
| Age | | | | | | |
| 22-30 | 323 | (3.3%) | 34 | (0.6%) | 289 | (6.1%) |
| 31-40 | 451 | (4.6%) | 60 | (1.2%) | 391 | (8.3%) |
| 41-50 | 805 | (8.3%) | 139 | (2.7%) | 666 | (14.1%) |
| 51-60 | 1483 | (15.3%) | 416 | (8.3%) | 1067 | (22.7%) |
| 61-70 | 2309 | (23.8%) | 1149 | (22.9%) | 1160 | (24.7%) |
| 71-80 | 2293 | (23.6%) | 1674 | (33.4%) | 619 | (13.1%) |
| 81+ | 2028 | (20.9%) | 1528 | (30.5%) | 500 | (10.6%) |
| Ethnicity | | | | | | |
| White | 8852 | (91.1%) | 4682 | (93.6%) | 4170 | (89.2%) |
| Black | 271 | (2.8%) | 91 | (1.8%) | 180 | (3.8%) |
| Asian | 174 | (1.7%) | 71 | (1.4%) | 103 | (2.2%) |
| Others | 376 | (3.8%) | 156 | (3.1%) | 220 | (4.7%) |

Furthermore, a significant proportion (68%) of individuals in the control group had been diagnosed with cancer, emphasizing the prevalence and impact of oncological diseases among adults attending the Mayo Clinic.

Table 4 shows individuals assessed using the Sokolow-Lyon Criteria, 11.34% tested positive, indicating a potential presence of left ventricular hypertrophy (LVH). Specifically, within this group, 16.22% of the positive cases were identified in the test group, while 6.46% were found in the control group

TABLE 4

| | Overall | Test (AVS) | Control |
|---|---|---|---|
| Sokolow-Lyon | 1134 (11.34%) | 811 (16.22%) | 323 (6.46%) |
| Criteria | | | |
| Cornell criteria | 877 (8.77%) | 657 (13.14%) | 220 (4.4%) |

Table 4 shows individuals assessed using the Sokolow-Lyon Criteria, 11.34% tested positive, indicating a potential presence of left ventricular hypertrophy (LVH). Specifically, within this group, 16.22% of the positive cases were identified in the test group, while 6.46% were found in the control group.

Comparatively, the Cornell Criteria identified a slightly lower prevalence of positive cases, with 8.77% of individuals testing positive for potential LVH. Within this cohort, 13.14% of the positive cases were detected among individuals diagnosed with atrioventricular septal defects (AVS), while 4.4% were found in the control group.

### Model Training

The model was trained on 9673 patients (see Table 2). The training dataset comprises parameters derived from all 12-lead ECG recordings. The training process involved leveraging labelled data from Mayo clinic to enable accurate detection and analysis of the desired patterns in the ECG signals related with AVS.

The table 5 below displays the parameters that were supplied to the model in a specific sequence. There are 62 parameters which are fed to the model which includes age of the patients, P wave amplitude, R wave amplitude, R wave duration, S wave amplitude, S wave duration, T wave amplitude of all the 12 leads and average QT.

TABLE 5

| Column name | Description |
|---|---|
| NFER_AGE | Patient's age (in years) |
| P_A_I | Pwave amplitude of lead I |
| P_A_II | Pwave amplitude of lead II |
| P_A_III | Pwave amplitude of lead III |
| P_A_V1 | Pwave amplitude of lead V1 |
| P_A_V2 | Pwave amplitude of lead V2 |
| P_A_V3 | Pwave amplitude of lead V3 |
| P_A_V4 | Pwave amplitude of lead V4 |
| P_A_V5 | Pwave amplitude of lead V5 |
| P_A_V6 | Pwave amplitude of lead V6 |
| P_A_aVF | Pwave amplitude of lead aVF |
| P_A_aVR | Pwave amplitude of lead aVR |
| P_A_aVL | Pwave amplitude of lead aVL |
| R_A_I | Rwave amplitude of lead I |
| R_A_II | Rwave amplitude of lead II |
| R_A_III | Rwave amplitude of lead III |
| R_A_V1 | Rwave amplitude of lead V1 |
| R_A_V2 | Rwave amplitude of lead V2 |
| R_A_V3 | Rwave amplitude of lead V3 |
| R_A_V4 | Rwave amplitude of lead V4 |
| R_A_V5 | Rwave amplitude of lead V5 |
| R_A_V6 | Rwave amplitude of lead V6 |
| R_A_aVF | Rwave amplitude of lead aVF |
| R_A_aVR | Rwave amplitude of lead aVR |
| R_A_aVL | Rwave amplitude of lead aVL |
| R_D_I | Rwave duration of lead I |
| R_D_II | Rwave duration of lead II |
| R_D_III | Rwave duration of lead III |
| R_D_V1 | Rwave duration of lead V1 |
| R_D_V2 | Rwave duration of lead V2 |
| R_D_V3 | Rwave duration of lead V3 |
| R_D_V4 | Rwave duration of lead V4 |
| R_D_V5 | Rwave duration of lead V5 |
| R_D_V6 | Rwave duration of lead V6 |
| R_D_aVF | Rwave duration of lead aVF |
| R_D_aVR | Rwave duration of lead aVR |
| R_D_aVL | Rwave duration of lead aVL |
| S_A_I | Swave amplitude of lead I |
| S_A_II | Swave amplitude of lead II |
| S_A_III | Swave amplitude of lead III |
| S_A_V1 | Swave amplitude of lead V1 |
| S_A_V2 | Swave amplitude of lead V2 |
| S_A_V3 | Swave amplitude of lead V3 |
| S_A_V4 | Swave amplitude of lead V4 |
| S_A_V5 | Swave amplitude of lead V5 |
| S_A_V6 | Swave amplitude of lead V6 |
| S_A_aVF | Swave amplitude of lead aVF |
| S_A_aVR | Swave amplitude of lead aVR |
| S_A_aVL | Swave amplitude of lead aVL |
| T_A_I | Twave amplitude of lead I |
| T_A_II | Twave amplitude of lead II |
| T_A_III | Twave amplitude of lead III |
| T_A_V1 | Twave amplitude of lead V1 |
| T_A_V2 | Twave amplitude of lead V2 |
| T_A_V3 | Twave amplitude of lead V3 |
| T_A_V4 | Twave amplitude of lead V4 |
| T_A_V5 | Twave amplitude of lead V5 |
| T_A_V6 | Twave amplitude of lead V6 |
| T_A_aVF | Twave amplitude of lead aVF |
| T_A_aVR | Twave amplitude of lead aVR |
| T_A_aVL | Twave amplitude of lead aVL |
| QT | QT interval |

The model learns to understand the nuances of the parameters and utilize that understanding to classify inputs into either of two (binary classification). The model's compilation is configured to use binary cross-entropy loss, the Adam optimizer, and accuracy as the evaluation metric. The training process incorporates two essential callbacks. The ReduceLROnPlateau callback adjusts the learning rate dynamically based on the plateauing of a monitored metric, which can aid in smoother convergence. The EarlyStopping callback prevents overfitting by monitoring validation metrics; if no significant improvement occurs within a defined patience period, training halts. The model is then trained for 1000 epochs with these callbacks guiding the process to enhance efficiency and prevent overfitting.

The below code shows the snippet for training.
model.compile(loss=‘binary_crossentropy’, optimizer=‘adam’, metrics=[‘accuracy’])
reduce_Ir=ReduceLROnPLATEAU( )
early_stopping=EarlyStopping (patience=50, min_delta=0.0001)
model.fit(x,y,epochs=1000, validation_data, callbacks=[reduce_Ir, early_stopping]
loss=‘binary_crossentropy’: This parameter specifies the loss function that the model will use during training. In this case, “binary_crossentropy” is employed. This is a suitable choice for binary classification tasks, as it quantifies the disparity between the model's predictions and the true binary labels, guiding the optimization process to minimize this discrepancy.
optimizer=‘adam’: The “optimizer” parameter designates the optimization algorithm responsible for updating the model's weights during training. Here, the Adam optimizer is utilized. Adam adapts the learning rate individually for each parameter, offering a dynamic approach that balances stability and speed in weight updates. This aids in expediting convergence while mitigating the chances of getting stuck in local minima.
metrics=[‘accuracy’]: This parameter specifies the evaluation metric(s) used to assess the model's performance during and after training. In this case, “accuracy” is chosen. Accuracy measures the ratio of correctly predicted instances to the total instances, providing insight into the model's ability to make correct binary classifications.
reduce_Ir=ReduceLROnPlateau( ) This callback monitors a specific metric (typically validation loss) during training. If the monitored metric plateaus for a specified number of epochs, the callback reduces the learning rate. This dynamic adjustment aims to aid convergence when progress becomes stagnant, allowing the model to fine-tune its behavior as it approaches a potential optimal solution.
early_stopping=EarlyStopping (patience=50, min_delta=0.0001): The EarlyStopping callback is employed to prevent overfitting. It watches a chosen validation metric and halts training if the metric fails to demonstrate significant improvement over a certain number of epochs, indicated by the “patience” parameter. The “min delta” parameter determines the minimal change in the monitored metric that qualifies as an improvement, setting a threshold.
model.fit (x, y, validation_data epochs=1000, callbacks=[reduce_Ir, early_stopping]): The fit function initiates the training process. It takes in the pre-processed input data x and target labels y. The “epochs” parameter specifies the number of training iterations. The “callbacks” parameter is used to provide the callbacks created earlier, namely reduce_Ir and early_stopping. These callbacks come into play during training to adjust the learning rate and possibly halt training early based on predefined conditions, ultimately enhancing the model's convergence and generalization.

After the training process is complete, the final set of weights, representing the weights acquired by the model during training, are saved. These saved weights can be easily loaded and utilized by the system for any further analysis of the AVS detection. This comprises the analysis module used in systems of the disclosure.

### Model Architecture

The AVS model is a feedforward neural network, a fundamental and versatile architecture often used for various machine learning tasks, particularly with structured or tabular data. This network consists of multiple fully connected layers (referred to as Dense layers in Keras), which play a crucial role in capturing complex patterns and relationships within the input data.

Each component in the network FIG. 2 serves a specific purpose. The input layer (‘inputs’) serves as the entry point for the normalized data, with its shape determined by the number of features in the input dataset. The following hidden layers (‘FC1’, ‘FC2’, ‘FC3’) are responsible for processing and transforming the data. These layers are characterized by their fully connected nature, meaning each neuron is connected to every neuron in the previous and subsequent layers.

To enhance training stability and convergence, batch normalization (‘BC1’, ‘BC2’, ‘BC3’) is applied after each fully connected layer. Batch normalization helps to mitigate issues like vanishing or exploding gradients by standardizing the input to each layer, making the training process more efficient. Furthermore, the Rectified Linear Unit (ReLU) activation functions (‘Activation1’, ‘Activation2’) introduce non-linearity, enabling the network to model complex, non-linear relationships in the data. The inclusion of dropout layers (‘Dropout1’, ‘Dropout2’, ‘Dropout3’) with a dropout rate of 0.3, 0.5, 0.3 respectively after some of the hidden layers is intended to prevent overfitting. Dropout randomly deactivates a fraction of neurons during training, promoting better generalization to unseen data.

The final layer (‘OutLayer’) plays a significant role in the model's goal, which is binary classification. This fully connected layer produces a single output, which is then passed through a sigmoid activation function (‘sigmoid’). The sigmoid function squashes the output into the range [0, 1], making it suitable for binary classification tasks. The output represents the predicted probability of the input data belonging to the positive class (1). FIG. 2 is a flow chart with illustrative steps of the feed-forward neural network deployed in developing the model.

The Table 6 below summarizes the details of the model:

TABLE 6

| Layer Name | Output Shape | Parameters |
|---|---|---|
| inputs | [(None, 62)] | 0 |
| FC1 | (None, 64) | 4032 |
| BC1 | (None, 64) | 256 |
| Activation1 | (None, 64) | 0 |
| Dropout1 | (None, 64) | 0 |
| FC2 | (None, 64) | 4160 |
| BC2 | (None, 64) | 256 |
| Activation2 | (None, 64) | 0 |
| Dropout2 | (None, 64) | 0 |
| FC3 | (None, 64) | 4160 |
| BC3 | (None, 64) | 256 |
| Dropout3 | (None, 64) | 0 |
| OutLayer | (None, 1) | 65 |
| sigmoid | (None, 1) | 0 |

The model architecture comprises an exemplary analysis module used the a system of the disclosure. It has 14 layers and 13185 parameters. The network function receives measurement matrices of 62 parameters and produces a parameter output between 0 and 1.

Insight into the Model Architecture Using SHAP.

SHAP (SHapley Additive explanations) is a powerful tool for explaining the predictions of machine learning models, including neural networks. It provides insights into how specific features influence the model's output and helps to interpret the black-box nature of complex models.

This technique can shed light on the often complex and nonlinear relationships within such neural networks, making them more interpretable and actionable.

SHAP operates by providing SHAP values for each feature in the dataset. For a given prediction, SHAP values represent the contribution of each feature to the prediction compared to the average prediction across all possible subsets of features. In the context of this feedforward neural network, SHAP values reveals how specific neurons in the layers respond to input features and how these responses propagate through the network. This information helps to identify which features play a more significant role in driving the model's decisions, providing valuable insights for feature selection, model fine-tuning, and understanding the neural network's inner workings.

The magnitude (absolute value) of the mean SHAP value reflects the strength of the feature's impact. A larger magnitude indicates a stronger influence on the model's predictions.

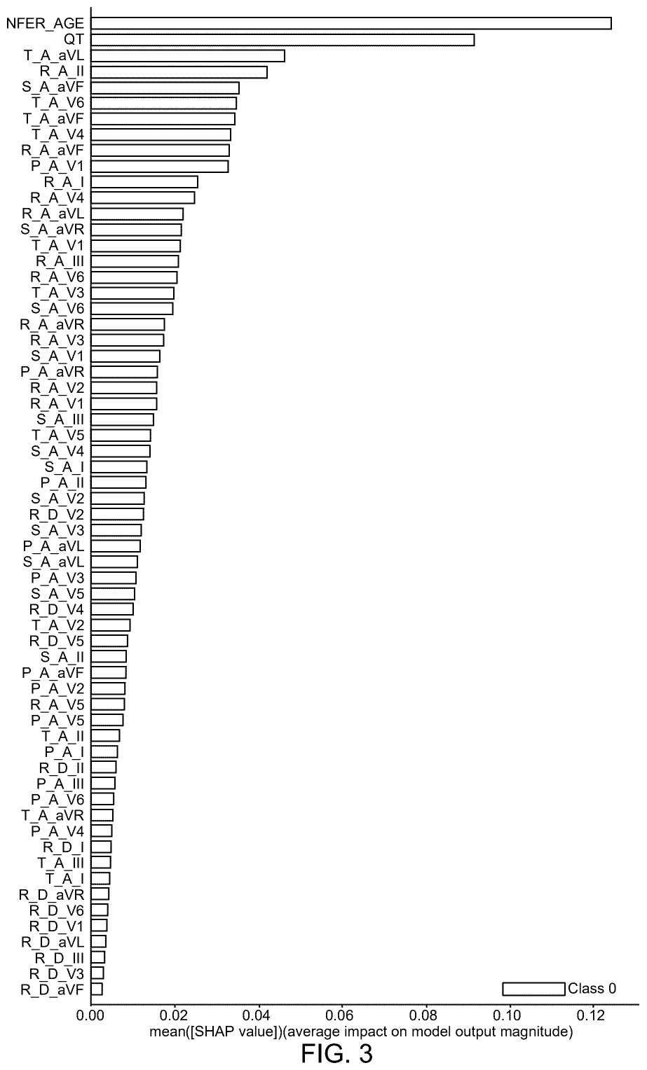

The SHAP analysis (FIG. 3) conducted has yielded significant insights into the influence of different features on the model's predictions. It was observed that variables such as AGE and QT interval exerted a substantial impact across the dataset, showcasing a remarkable degree of influence on the predictive outcomes. These findings underscore the importance of considering demographic factors and cardiac health indicators in assessing predictive models' performance and reliability. The robust influence of AGE highlights the significance of age-related physiological changes in cardiac health, whereas the QT interval's prominence underscores its clinical relevance as a measure of ventricular depolarization and repolarization. The mean SHAP value for AGE averaged around 0.12, emphasizing its contribution to predictive outcomes.

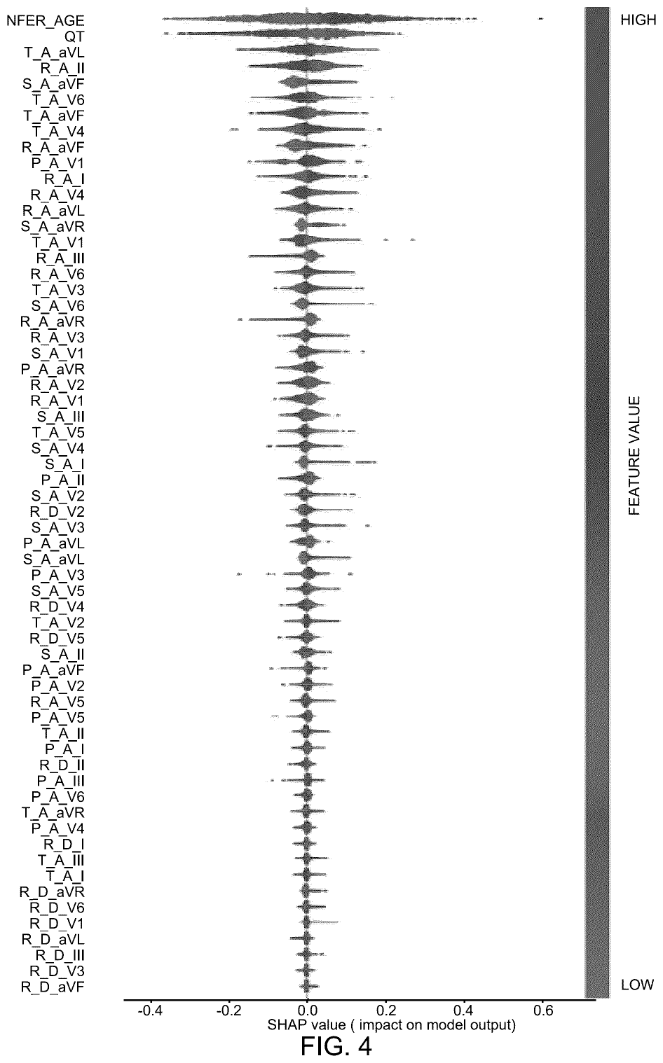

The bee swarm diagram (FIG. 4) showcasing SHAP values derived from a unified model, serves as a potent visual aid for understanding the dynamics of predictive features. Upon closer inspection, it becomes evident that as the age of individuals within the dataset increases, the corresponding impact on the model's predictions becomes more pronounced. This observation hints at the pivotal role age plays in shaping the model's output, suggesting a heightened influence exerted by older individuals on the predicted outcomes. This finding underscores the importance of considering age as a critical factor in predictive modeling endeavors, particularly within the realm of healthcare where age-related physiological changes can significantly impact prognosis and risk assessment. Moreover, the discernible increase in the model's sensitivity to age highlights the necessity of integrating age-related considerations into predictive frameworks, especially when dealing with conditions or phenomena known to exhibit age-dependent patterns.

The observations drawn from the bee swarm diagram regarding the QT interval further unveil the intricate relationship between this cardiac parameter and the model's predictive outcomes. As the QT interval decreases, distinct patterns emerge in the impact of various features on the model's predictions, shedding light on their dynamic interplay. Notably, the T amplitude from lead V4 (T A v4) exhibits a notable increase, suggesting that lower QT intervals are associated with higher values of T amplitude in this specific lead. This finding underscores the potential role of the QT interval in modulating the amplitude of T waves in certain electrocardiographic leads, which could have clinical implications for identifying cardiac abnormalities. This was surprising and unexpected.

Contrasting trends are observed in other features, such as T amplitude from lead AVL (T A AVL) and R amplitude from lead II (R A lead II), which demonstrate a decrease as the QT interval decreases.

Also, the bee swarm diagram reveals an increase in R amplitude from lead AVL (R A AVL) as the QT interval decreases, suggesting a positive association between QT interval decrease and R amplitude increase in this lead. This observation adds another layer of complexity to the understanding of QT interval dynamics and their implications for cardiac health. The positive correlation between QT interval decrease and R amplitude increase in lead AVL underscores the importance of considering lead-specific variations in electrocardiographic parameters when interpreting cardiac function.

### Model Validation:

The validation dataset was curated from a repository of ECG recordings of adult patients receiving medical care at the Mayo Clinic over a substantial period, spanning from 2017 to 2022. The dataset specifically focuses on patients who meet distinct criteria and showcases the outcomes of a comprehensive study. Within this cohort, we homed in on patients aged 22 and above who exhibited signs of AVS.

To ensure the dataset's quality and relevance, two fundamental conditions were imposed for patient inclusion for the test group. Firstly, patients had to have undergone at least one echocardiogram during their visit to Mayo Hospital. This echocardiogram is a pivotal diagnostic tool for evaluating the health of the heart. Secondly, their medical records needed to contain a diagnostic code that indicated the presence of any form of AVS, ensuring that patients with potential cardiac health issues related to AVS were included. This validation dataset consists of the remaining 7282 patients with AVS who were not utilized in the model training phase. In addition to AVS, these patients also presented with other comorbidities:
Approximately 38% of the patients had experienced a myocardial infarction (MI), indicating a history of heart attacks;
Around 43% of the patients had chronic kidney disease (CKD), suggesting impaired kidney function as an additional health concern;
Nearly half of the patients (51%) had been diagnosed with heart failure, indicating a significant burden on the heart's pumping ability;
A small percentage (5%) of patients had inflammatory heart diseases, indicating underlying inflammation affecting the heart;
Some patients (2.4%) had metabolic heart diseases, which might involve disorders related to how the body processes energy;
A substantial portion (79%) of the patients had a history of cancer, indicating the presence of malignancies alongside cardiovascular issues;
About 16% of patients had cardiomyopathy, which involves abnormalities in the heart muscle structure or function.

To establish a comparative framework for the study, three control groups were chosen. These control groups were drawn from the same original pool of adult patients who sought medical treatment at the Mayo Clinic between 2017 and 2022 and were aged 22 or above.

First control group individuals who did not undergo an echocardiogram during their visit to the Mayo Clinic. Additionally, these patients did not receive a clinical diagnosis of any form of AVS. This careful selection process ensures that the control group consists of individuals without any known health concerns related to AVS. To enable a comprehensive comparison, the most recent electrocardiogram (ECG) data was collected for these control group patients. This allows for a thorough evaluation of the differences and patterns between patients with AVS-related health concerns and those without, providing valuable insights for the study. 57636 patients were selected in this control group for validation.

The second control group, participants may or may not have hypertension, but those with a pacemaker are excluded to maintain consistency with the other groups. Similar to the test and Control Group 2, ECG data collected from January 2017 onwards are included for analysis, and all participants are aged 22 years or older. Additionally, all individuals in this group have undergone an echocardiogram, with data pairs collected approximately ±180 days apart to provide a comprehensive understanding of cardiac function over time. 106,019 patients were selected in this control group for validation. Exclusion criteria in this group are focused on individuals with valvular diseases, pacemaker, ensuring a homogenous study population.

Approximately 20% of the patients had experienced a myocardial infarction (MI), indicating a history of heart attacks;

Around 21% of the patients had chronic kidney disease (CKD), suggesting impaired kidney function as an additional health concern;

Nearly half of the patients (20%) had been diagnosed with heart failure, indicating a significant burden on the heart's pumping ability;

A small percentage (3%) of patients had inflammatory heart diseases, indicating underlying inflammation affecting the heart;

Some patients (2.6%) had metabolic heart diseases, which might involve disorders related to how the body processes energy;

A substantial portion (69%) of the patients had a history of cancer, indicating the presence of malignancies alongside cardiovascular issues;

About 8% of patients had cardiomyopathy, which involves abnormalities in the heart muscle structure or function.

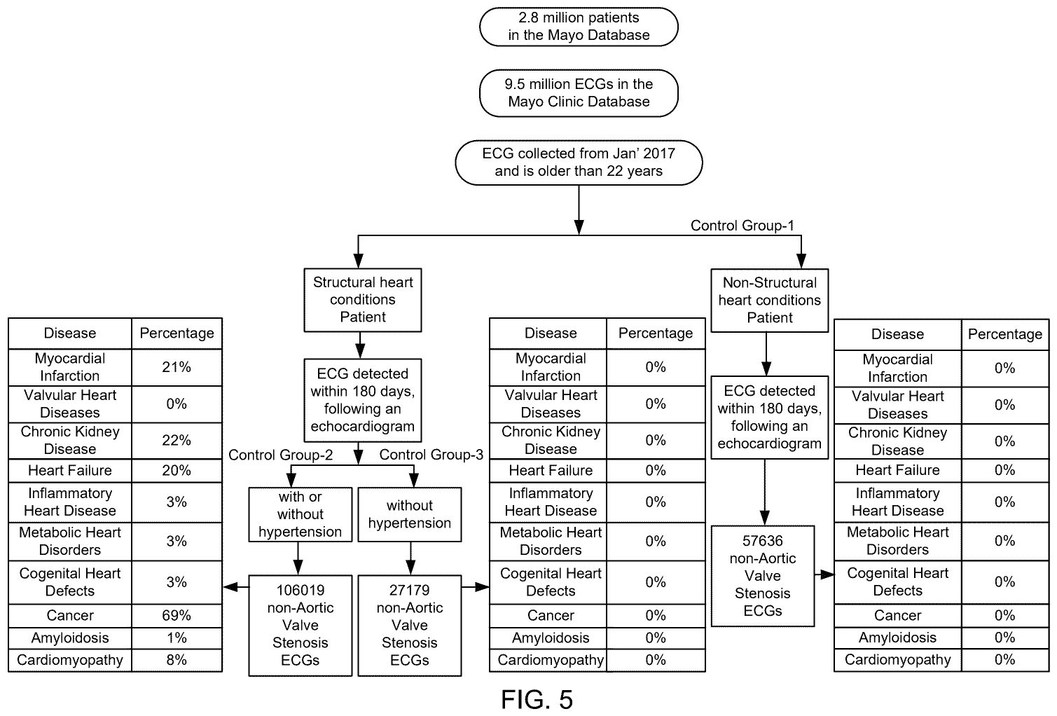

Third control group consist of participants who have undergone an echocardiogram but do not have hypertension. It does control patients of control group 2. Similar to the other groups, ECG data are collected from January 2017 onwards, ensuring consistency across all groups. Additionally, participants in this group are aged 22 years or older. 27,179 patients were selected in this control group for validation of which 17,236 patients are from control group 2. Exclusion criteria are comprehensive, ensuring individuals with a wide range of cardiovascular conditions, such as valvular diseases, heart failure, chronic kidney disease, inflammatory heart diseases, amyloidosis, cardiomyopathy, abnormal ECG or echocardiogram results, aortic valve stenosis, pacemaker implantation, valve replacement, and heart transplant or surgery, are excluded. FIG. 5 is a flow chart depicting steps for the creation of the validation dataset.

Table 7 provides an overview of the patient demographics within the Validation dataset. A sample of 198,116 was collected for validation. In this dataset 48% of the patients were identified as male and 52% as female. The data collection process spanned through all the mayo hospitals including Rochester, Arizona, Florida, and Mayo Clinic Hospital System (MCHS).

TABLE 7

| | Overall | | Test | | Control (C1) | | Control (C2) | | Control (C3) | |
|---|---|---|---|---|---|---|---|---|---|---|
| Patients | 198116 | | 7282 | | 57636 | | 27179 | | 106019 | |
| count | | | | | | | | | | |
| Gender | | | | | | | | | | |
| Male | 94695 | (47.79) | 4142 | (56.88%) | 26222 | (45.49%) | 12000 | (44.15%) | 52331 | (49.36%) |
| Female | 103403 | (52.19) | 3140 | (43.12%) | 31403 | (54.49%) | 15178 | (55.84%) | 53682 | (50.63%) |
| Age (Mean) | 57.15 | | 76.26 | | 53.93 | | 52.38 | | 58.81 | |
| 22-30 | 13988 | (7.06) | 52 | (0.71%) | 5995 | (10.40%) | 2387 | (8.78%) | 5554 | (5.24%) |
| 31-40 | 21488 | (10.85) | 87 | (1.19%) | 7964 | (13.82%) | 3946 | (14.52%) | 9491 | (8.95%) |
| 41-50 | 30408 | (15.35) | 182 | (2.49%) | 9549 | (16.57%) | 5456 | (20.07%) | 15221 | (14.36%) |
| 51-60 | 43998 | (22.21) | 643 | (8.83%) | 12223 | (21.21%) | 6880 | (25.31%) | 24252 | (22.88%) |
| 61-70 | 49054 | (24.76) | 1532 | (21.04%) | 13264 | (23.01%) | 5805 | (21.36%) | 28453 | (26.84%) |
| 71-80 | 29166 | (14.72) | 2523 | (34.65%) | 6864 | (11.91%) | 2291 | (8.43%) | 17488 | (16.49%) |
| 81+ | 9981 | (5.04) | 2263 | (31.08%) | 1777 | (3.08%) | 381 | (1.40%) | 5560 | (5.24%) |
| Race | | | | | | | | | | |
| White | 174486 | (88.07) | 6810 | (93.52%) | 50889 | (88.29%) | 23799 | (87.56%) | 92988 | (87.71%) |
| Black | 9157 | (4.62) | 143 | (1.96%) | 2602 | (4.51%) | 1022 | (3.76%) | 5390 | (5.08%) |
| Asian | 4814 | (2.43) | 91 | (1.25%) | 1472 | (2.55%) | 735 | (2.70%) | 2516 | (2.37%) |

| | | | | | | | | | |
|---|---|---|---|---|---|---|---|---|---|
| Others | 9659 | (4.88) | 238 | (3.27%) | 2673 | (4.64%) | 1623 | (5.97%) | 512583%) |

In the validation dataset of patients with AVS, the gender distribution reflects a predominance of male patients, constituting 56% of the group, while female patients make up the remaining 43%. The average age among these individuals stands at 76 years. Notably, a significant proportion of this cohort, specifically 93%, are white.

Conversely, the control group 1 with no echocardiogram exhibits a distinct demographic profile. Within this group, male individuals account for 45% of the cases, and female individuals make up 54%, The average age of individuals in the control group is notably lower, averaging 53 years. Also, the control group is predominantly white, comprising 88% of the group.

Control group 2 exhibits a distinct demographic profile. Within this group, male individuals account for 49% of the cases, and female individuals make up 50%, The average age of individuals in the control group is notably lower, averaging 58 years. Also, 88% of the control group is white.

The control group 3 exhibits a distinct demographic profile. Within this group, male individuals account for 44% of the cases, and female individuals make up 55%, The average age of individuals in the control group is notably lower, averaging 52 years. Also, 87% of the control group is white.

### Model Performance:

A sample size of 198,116 ECG data was used for validation. The model was evaluated at 2 operating points selected from the development data set, one selected for equal sensitivity and specificity and the other for high (85%) sensitivity. These thresholds were applied to the validation data sets to characterize the sensitivity and specificity of the algorithm. Exact 95% confidence intervals were used for all measures of diagnostic performance.

### Case Studies and Results:

The algorithm performed well in identifying AS in the validation data sets.

TABLE 8

| | OVERALL COUNT | | |
|---|---|---|---|
| | 64918 | 113301 | 34461 |
| | Case | Case | Case |
| | Study-1 | Study -2 | Study-3 |
| Sokolow-Lyon | 4090 (6.3%) | 8941 (7.89%) | 2303 (6.68%) |
| Criteria | | | |
| Cornell criteria | 2659 (4.09%) | 6382 (5.63%) | 1518 (4.40%) |

The above table 8 shows overall count of individuals assessed across three distinct case studies reveals varying prevalence rates when employing both the Sokolow-Lyon Criteria and the Cornell Criteria for detecting left ventricular hypertrophy (LVH).

### Case-Study 1:

Case study is conducted on test group vs control group 1, comprising a total population of 64,918 individuals. The Sokolow-Lyon Criteria identified 4,090 individuals (6.3%) as testing positive for potential left ventricular hypertrophy (LVH). Concurrently, the Cornell Criteria detected positive results in 2,659 individuals (4.09%).

The result is as below.

The test (Table 9) demonstrates a sensitivity of 73.97% (with a confidence interval between 72.96% and 74.98%) and a specificity of 80.62% (ranging from 80.3% to 80.94%). The positive predictive value (PPV) is 32.54% (with a confidence interval from 31.83% to 33.25%), while the negative predictive value (NPV) is notably higher at 96.08% (ranging from 95.91% to 96.25%). The overall accuracy of the test in this scenario is 79.87%.

When sensitivity equals specificity (SE=SP), both sensitivity and specificity are 77.45% (with confidence intervals of 76.49%-78.41% and 77.11%-77.79% respectively). PPV and NPV are 30.27% (ranging from 29.61% to 30.93%) and 96.45% (ranging from 96.28% to 96.62%) respectively. The accuracy remains the same at 77.45%.

When (SE=85%), the sensitivity is increased to 85.0% (with a confidence interval from 84.18% to 85.82%), while the specificity decreases to 68.8% (ranging from 68.42% to 69.18%). Consequently, the PPV decreases to 25.61% (ranging from 25.06% to 26.16%), but the NPV increases to 97.32% (ranging from 97.16% to 97.48%). The overall accuracy in this scenario is 70.62%.

### Case-Study 2 (CS2)

Case Study 2 is done between test group and control Group2. In Case Study 2, 113,301 individuals, the prevalence of positive findings increased slightly for both criteria. Specifically, 8,941 individuals (7.89%) tested positive for potential LVH based on the Sokolow-Lyon Criteria, while 6,382 individuals (5.63%) tested positive using the Cornell Criteria.

The test (Table 9) demonstrates a sensitivity of 73.98% (with a confidence interval ranging from 72.97% to 74.99%) and a specificity of 67.32% (ranging from 67.04% to 67.60%). The positive predictive value (PPV) is relatively low at 13.45% (with a confidence interval from 13.12% to 13.78%), while the negative predictive value (NPV) is notably higher at 97.41% (ranging from 97.29% to 97.53%). The overall accuracy of the test in this scenario is 67.74%.

When sensitivity equals specificity (SE=SP), both sensitivity and specificity are 70.37% (with confidence intervals of 69.32%-71.42% and 70.1%-70.64% respectively). PPV and NPV are 14.02% (ranging from 13.66% to 14.38%) and 97.19% (ranging from 97% to 97.31%) respectively. The accuracy remains the same at 70.37%.

When the sensitivity increases to 85% (SE=85%), with a confidence interval from 84.18% to 85.82%), while the specificity decreases to 53.78% (ranging from 53.48% to 54.08%). Consequently, the PPV decreases to 11.22% (ranging from 10.96% to 11.48%), but the NPV increases to 98.12% (ranging from 98% to 98.23%). The overall accuracy in this scenario is 55.79%.

### Case-Study 3 (CS3)

Case study 3 is conducted between test group and control Group 3. In Case Study 3, 34,461 individuals, 2,303 (6.68%) tested positive using the Sokolow-Lyon Criteria, and 1,518 (4.40%) tested positive using the Cornell Criteria.

The test (Table 9) exhibits a sensitivity of 73.98% (with a confidence interval ranging from 72.97% to 74.99%) and a specificity of 85.56% (ranging from 85.14% to 85.98%). The positive predictive value (PPV) and negative predictive value (NPV) are 57.86% (with a confidence interval from 56.86% to 58.86%) and 92.47% (ranging from 92.14% to 92.8%) respectively. The overall accuracy of the test in this scenario is 83.12%.

In the SE=SP scenario, both sensitivity and specificity are set to 80.25% (with confidence intervals of 79.34%-81.16% and 79.78%-80.72% respectively). The PPV and NPV are 52.13% (ranging from 51.21% to 53.05%) and 93.85% (ranging from 93.54% to 94.16%) respectively. The accuracy remains consistent at 80.25%.

In the SE=85% scenario, the sensitivity is increased to 85.0% (with a confidence interval from 84.18% to 85.82%), while the specificity decreases to 75.39% (ranging from 74.88% to 75.9%). Consequently, the PPV decreases to 48.06% (ranging from 47.2% to 48.92%), but the NPV increases to 94.94% (ranging from 94.65% to 95.23%). The overall accuracy in this scenario is 77.42%.

TABLE 9

| | | | | | |
|---|---|---|---|---|---|
| Description | SE | SP | PPV | NPV | Accuracy |

CS-1

| | | | | | |
|---|---|---|---|---|---|
| Result | 73.97 | 80.62 | 32.54 | 96.08 | 79.87% |
| | (72.96-74.98) | (80.3-80.94) | (31.83-33.25) | (95.91-96.25) | |
| SE = SP | 77.45 | 77.45 | 30.27 | 96.45 | 77.45% |
| | (76.49-78.41) | (77.11-77.79) | (29.61-30.93) | (96.28-96.62) | |
| SE = 85% | 85.0 | 68.8 | 25.61 | 97.32 | 70.62% |
| | (84.18-85.82) | (68.42-69.18) | (25.06-26.16) | (97.16-97.48) | |

CS-2

| | | | | | |
|---|---|---|---|---|---|
| Result | 73.98 | 67.32 | 13.45 | 97.41 | 67.74% |
| | (72.97-74.99) | (67.04-67.60) | (13.12-13.78) | (97.29-97.53) | |
| SE = SP | 70.37 | 70.37 | 14.02 | 97.19 | 70.37% |
| | (69.32-71.42) | (70.1-70.64) | (13.66-14.38) | (97-97.31) | |
| SE = 85% | 85.0 | 53.78 | 11.22 | 98.12 | 55.79% |
| | (84.18-85.82) | (53.48-54.08) | (10.96-11.48) | (98-98.23) | |

CS-3

| | | | | | |
|---|---|---|---|---|---|
| Result | 73.98 | 85.56 | 57.86 | 92.47 | 83.12% |
| | (72.97-74.99) | (85.14-85.98) | (56.86-58.86) | (92.14-92.8) | |
| SE = SP | 80.25 | 80.25 | 52.13 | 93.85 | 80.25% |
| | (79.34-81.16) | (79.78-80.72) | (51.21-53.05) | (93.54-94.16) | |
| SE = 85% | 85.0 | 75.39 | 48.06 | 94.94 | 77.42% |
| | (84.18-85.82) | (74.88-75.9) | (47.2-48.92) | (94.65-95.23) | |

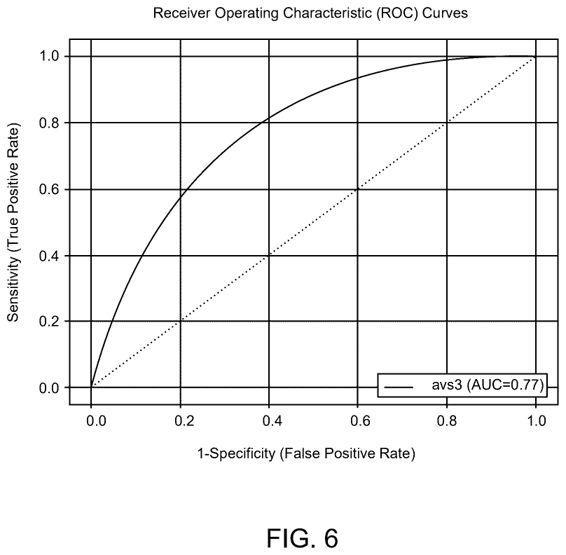

AUC indicates area under the receiver operating characteristic curve for all the test cases (see FIG. 6).

The number of false-positive, false-negative, true-positive, and true-negative results for each model, as well as accuracy, is presented in Table 10.

TABLE 10

| | | | | | |
|---|---|---|---|---|---|
| Description | TN | FP | FN | TP | Accuracy |

CS-1

| | | | | | |
|---|---|---|---|---|---|
| Result | 46466 | 11170 | 1895 | 5387 | 79.87% |
| SE = SP | 44641 | 12995 | 1642 | 5640 | 77.45% |
| SE = 85% | 39655 | 17981 | 1092 | 6190 | 70.62% |

CS-2

| | | | | | |
|---|---|---|---|---|---|
| Result | 71368 | 34651 | 1895 | 5387 | 67.74% |
| SE = SP | 74601 | 31418 | 2158 | 5124 | 70.37% |
| SE = 85% | 57022 | 48997 | 1092 | 6190 | 55.79% |

CS-3

| | | | | | |
|---|---|---|---|---|---|
| Result | 23256 | 3923 | 1895 | 5387 | 83.12% |
| SE = SP | 21812 | 5367 | 1438 | 5844 | 80.25% |
| SE = 85% | 20490 | 6689 | 1092 | 6190 | 77.42% |

Conclusion: The model proposed suggests integrating electrocardiography (ECG) as a complementary screening tool alongside LVH assessment to enhance diagnostic accuracy for AVS. By incorporating ECG parameters, such as ventricular hypertrophy patterns, arrhythmias, and conduction abnormalities, the model aims to provide a more comprehensive evaluation of cardiac function and structure. ECG offers valuable insights into electrical activity within the heart, which can help identify subtle abnormalities indicative of AVS, particularly in early stages when symptoms may be absent or nonspecific.

This integrated approach not only improves the detection of AVS but also enables risk stratification and facilitates timely intervention, ultimately enhancing patient outcomes. Moving forward, further validation and refinement of the proposed model through larger-scale studies and clinical trials are essential steps toward its integration into routine practice, potentially revolutionizing AVS screening and management strategies.

### Example 2—Predicting Aortic Stenosis in the Community Using an Artificial Intelligence Model for 12-Lead Electrocardiograms

This example details further validation of the processes and systems of the disclosure in effectively detecting moderate to severe aortic stenosis (AS) and risk of incident of heart failure (HF) in the community.

Methods: the processes and systems described in Example 1 were applied to participants free of cardiovascular (CV) disease and with available echocardiograms from visit 5 of the Atherosclerosis Risk in Community (ARIC) study. The model's performance was evaluated for predicting moderate/severe AS. Time to incident moderate/severe AS and incident HF were assessed using Cox proportional hazards models. Models were assessed unadjusted and adjusted for traditional CV risk factors (PCP-HF risk score).

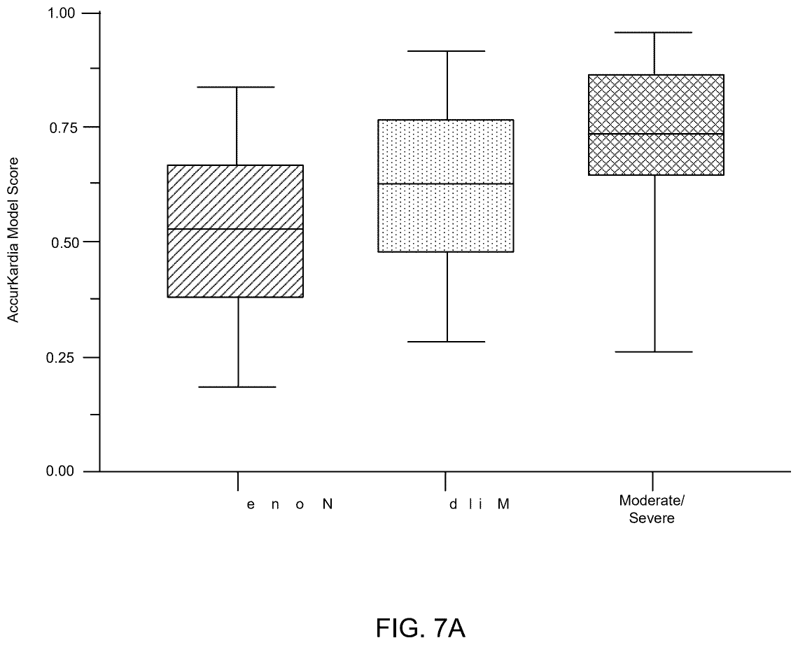

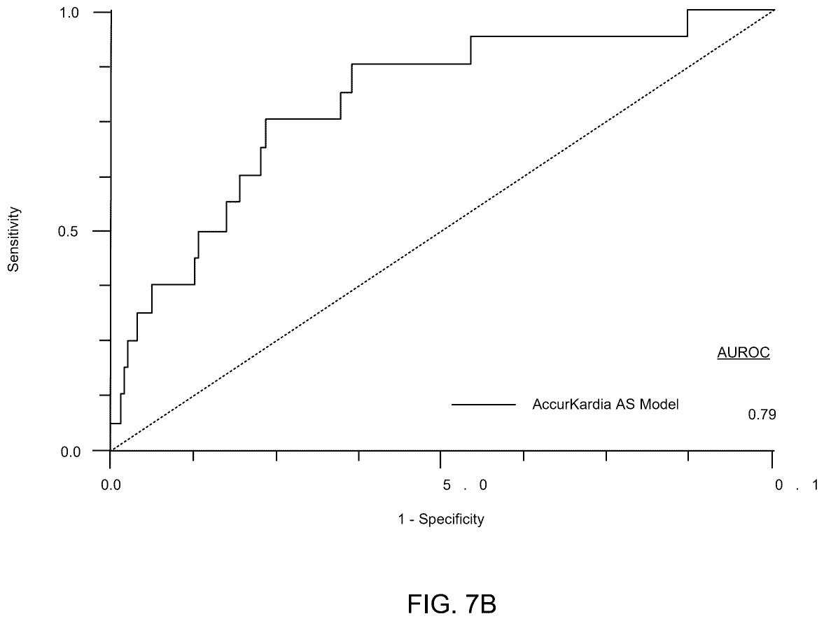

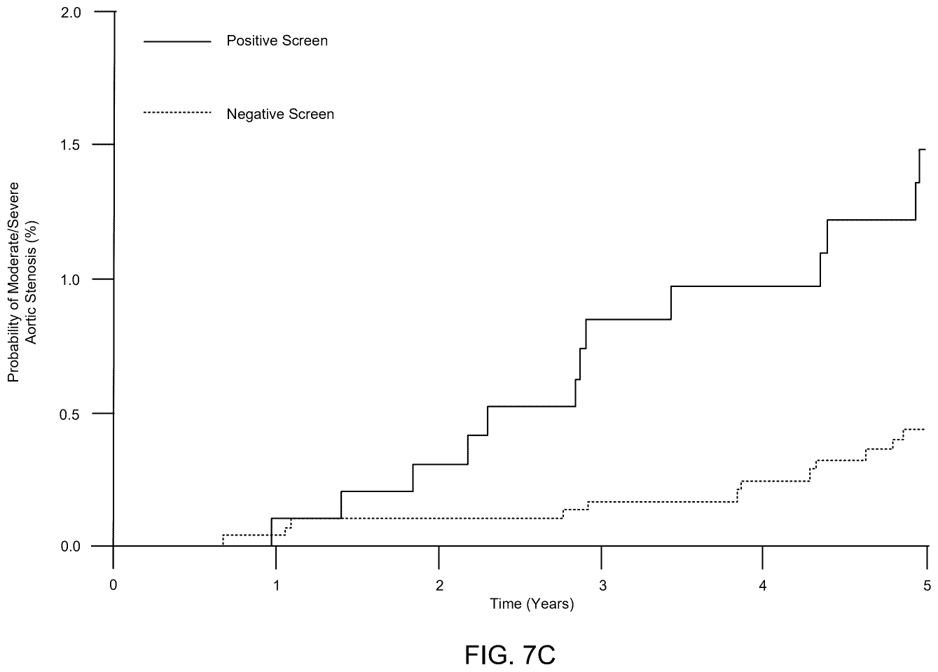

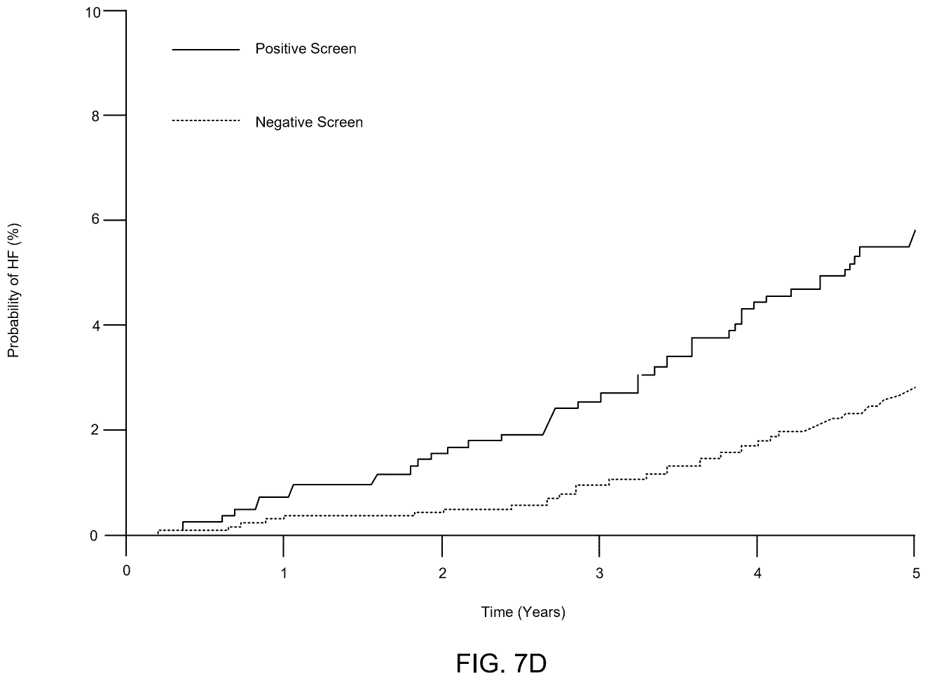

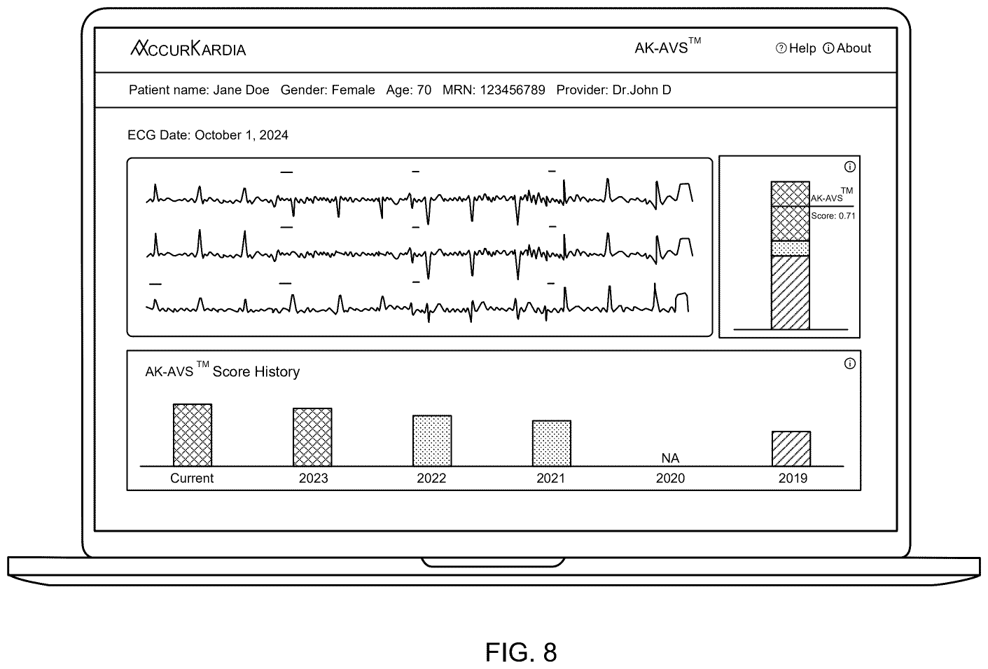

Results: 3,662 participants were included in the analysis. Amongst them 16 (0.4%) had moderate/severe AS and 126 (3.4%) had mild AS. The median score provided with the systems and processes of Example 1 was 0.54, 0.62, and 0.74 for normal, mild, and moderate/severe AS, respectively (FIG. 7A). The model demonstrated an AUROC of 0.79, sensitivity of 0.75, specificity of 0.77, PPV of 0.01, and NPV of 0.99 for predicting moderate/severe AS (FIG. 7B). Notably, false-positive predictions were associated with a significantly higher risk of developing moderate/severe AS within 5-years (HR 5.21, 95% CI 2.83-9.61, FIG. 7C) that remained significant after adjustment (aHR 4.41, 95% CI 2.29-8.50). Similar results were observed with time to incident HF (aHR 1.55, 95% CI 1.11-2.16, FIG. 7D). FIG. 8 (FIG. 8) is an illustration of an exemplary output of the analysis provided by the systems and processes of the disclosure.

Conclusion: The systems and processes described herein accurately predict moderate/severe AS and identifies individuals at higher risk for developing AS and HF in the ARIC community cohort, demonstrating its potential as a screening tool.

### Example 3—Prognostic Value of Periprocedural AI-ECG Score Changes in TAVR Recipients

This example illustrates the use of a process and a system ECG model (AK-AVS) to predict clinical outcomes following transcatheter aortic valve replacement (TAVR).

Methods: A retrospective analysis was conducted on 1,596 TAVR recipients. See FIG. 9 for the study design. ECGs were obtained immediately before and after TAVR and analyzed using the system and model described in the above examples. Baseline scores and post-TAVR score changes were correlated with clinical outcomes, including 1-year mortality, permanent pacemaker implantation, and length of hospital stay. Logistic regression and negative binomial regression were used, adjusting for clinical and procedural confounders.

### Results:

90.8% of patients exceeded an AK-AVS threshold of 0.5 at 6 months pre-TAVR; 82.4% exceeded 0.6; 50% exceeded 0.8.

Among 450 patients with 5+ ECGs prior to TAVR, AK-AVS scores >0.6 were detected 4.51 years before TAVR.

Three trajectory clusters were identified: Persistently High (57.1%), Accelerated Progression (23.6%), and Stable Low (19.3%). Persistently High and Accelerated Progression clusters independently predicted higher 1-year mortality (adjusted HR 1.48 and 1.40, respectively) compared to Stable Low.

Adding cluster information improved 1-year mortality risk prediction (EuroSCOREII+clusters: C-index=0.613 vs 0.607; STS+clusters: C-index=0.607 vs 0.580).

TABLE 11

| | Baseline Score from the Systems | | Change in Score | |
|---|---|---|---|---|
| | and Methods (ref: score <0.62) | | (ref: decrease in score) | |
| | Estimate (95% CI) | P-value | Estimate (95% CI) | P-value |
| 1-year Mortality | 1.20 (0.78, 1.84) | 0.41 | 1.78 (1.09, 2.91) | 0.02 |
| Pacemaker | 0.96 (0.71, 1.32) | 0.82 | 1.68 (1.18, 2.40) | 0.004 |
| Implant | | | | |
| Length of Stay | 0.90 (0.82, 0.98) | 0.01 | 1.17 (1.06, 1.28) | 0.001 |

Models estimated using logistic regression and expressed as odds ratio, except for length of stay which is modeled using negative-binomial regression and expressed as incidence rate ratios. All models adjusted for baseline AK-AVS score, STS mortality risk score, valve size, Agatston score, device implant type, and baseline QRS duration.
Abbreviations:
Aortic Valve Stenosis;
CI, confidence interval;
TAVR, transcatheter aortic valve replacement

Conclusion: Periprocedural changes in the AI-ECG score provide prognostic information, identifying patients at higher risk for adverse outcomes post-TAVR.

### Example 4—Longitudinal Trajectories for Early Identification of High-Risk Aortic Stenosis Phenotypes

This example demonstrates the use of longitudinal AI-ECG analysis to identify high-risk aortic stenosis (AS) phenotypes years before TAVR.

Methods: A total of 7,860 ECGs from 2,040 TAVR recipients were analyzed, spanning up to 10 years pre-procedure. The AK-AVS model was used to generate scores for each ECG. Unsupervised clustering identified distinct longitudinal score trajectories, which were correlated with clinical outcomes using Cox models adjusted for clinical factors. The predictive utility of adding trajectory clusters to standard risk scores (STS, EuroSCOREII) was evaluated.

### Results:

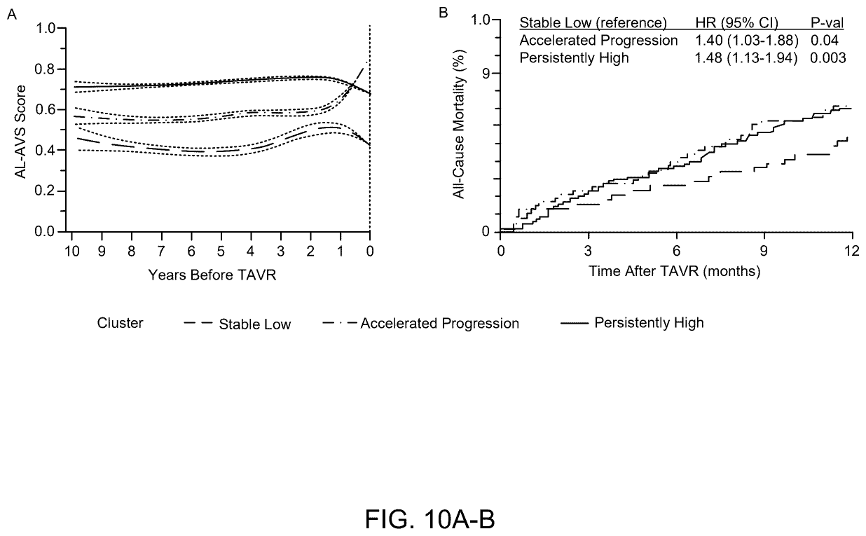

90.8% of patients exceeded an AK-AVS threshold of 0.5 at 6 months pre-TAVR; 82.4% exceeded 0.6; 50% exceeded 0.8. See FIG. 10A. Among 450 patients with 5+ ECGs prior to TAVR, AK-AVS scores >0.6 were detected 4.51 years before TAVR. See FIG. 10B.

Three trajectory clusters were identified: Persistently High (57.1%), Accelerated Progression (23.6%), and Stable Low (19.3%). Table 12 provides a summary of the baseline characteristics by trajectory cluster as identified by a system and a process o the disclosure.

TABLE 12

| | | | Accelerated | | Persistently | | |
|---|---|---|---|---|---|---|---|
| | Stable Low | | Progression | | High | | |
| | (n = 393) | | (n = 482) | | (n = 1165) | | P-value |
| Demographics | | | | | | | |
| Age, years | 72.6 ± 10.0 | | 78.4 ± 8.2 | | 77.8 ± 9.4 | | <0.001 |
| Male sex | 239 | (60.8) | 274 | (56.8) | 688 | (59.1) | 0.48 |
| BMI, kg/m² | 28.4 | [24.8-33.0] | 27.4 | [24.3-31.6] | 27.8 | [24.3-32.5] | 0.08 |
| EuroSCORE 11, % | 5.3 | [3.4, 7.6] | 6.3 | [4.4, 8.5] | 6.2 | [4.3, 8.7] | <0.001 |
| Valve type | | | | | | | 0.49 |
| Core Valve | 149 | (37.9) | 156 | (32.4) | 394 | (33.8) | |
| Balloon- | 244 | (62.1) | 326 | (67.6) | 771 | (66.2) | |
| expanding* | | | | | | | |
| Echocardiographic | | | | | | | |
| Parameters | | | | | | | |
| AVA, cm² | 0.80 ± 0.26 | | 0.76 ± 0.18 | | 0.77 ± 0.20 | | 0.01 |
| Mean gradient, | 39.1 ± 13.8 | | 39.4 ± 12.9 | | 39.8 ± 13.1 | | 0.61 |
| mmHg | | | | | | | |
| Peak velocity, mis | 4.0 ± 0.7 | | 4.2 ± 2.8 | | 4.1 ± 1.9 | | 0.54 |
| Ejection fraction, % | 60 | [49-60] | 60 | [50-60] | 60 | [50-60] | 0.24 |
| Agatston score | 2511 ± 1375 | | 2441 ± 1345 | | 2452 ± 1288 | | 0.69 |
| Comorbidities | | | | | | | |
| Coronary artery | 327 | (83.2) | 398 | (82.6) | 974 | (83.6) | 0.87 |
| disease | | | | | | | |
| Chronic kidney | 115 | (29.3) | 167 | (34.6) | 420 | (36.1) | 0.049 |
| disease | | | | | | | |
| Diabetes mellitus | 163 | (41.5) | 186 | (38.6) | 444 | (38.1) | 0.49 |
| Atrial fibrillation | 96 | (24.4) | 144 | (29.9) | 340 | (29.2) | 0.14 |
| Peripheral vascular | 224 | (57.0) | 252 | (52.3) | 675 | (57.9) | 0.10 |
| disease | | | | | | | |
| COPD | 155 | (39.4) | 183 | (38.0) | 423 | (36.3) | 0.50 |
| Pre-existing | 42 | (10.7) | 79 | (16.4) | 201 | (17.3) | 0.008 |

pacemaker

Values are mean ± SD, median (IQR), or n (%).
Abbreviations:
AVA = aortic valve area;
BMI = body mass index;
COPD = chronic obstructive pulmonary disease;
IQR = interquartile range;
KCCQ = Kansas City Cardiomyopathy Questionnaire;
SD = standard deviation;
STS = Society of Thoracic Surgeons;
TAVR = transcatheter aortic valve replacement

Persistently High and Accelerated Progression clusters independently predicted higher 1-year mortality (adjusted HR 1.48 and 1.40, respectively) compared to Stable Low.

Adding cluster information improved 1-year mortality risk prediction (EuroSCOREII+clusters: C-index=0.613 vs 0.607; STS+clusters: C-index=0.607 vs 0.580).

Conclusion: Longitudinal AI-ECG analysis detects AS-related changes up to 4.5 years before TAVR, with distinct trajectories predicting mortality, supporting its use for early detection and risk stratification.

### Example 5—Predictive Value of AK-AVS Score Trajectories and Early Detection

This example highlights the predictive value of AK-AVS score trajectories and the potential for early detection of moderate-to-severe AS.

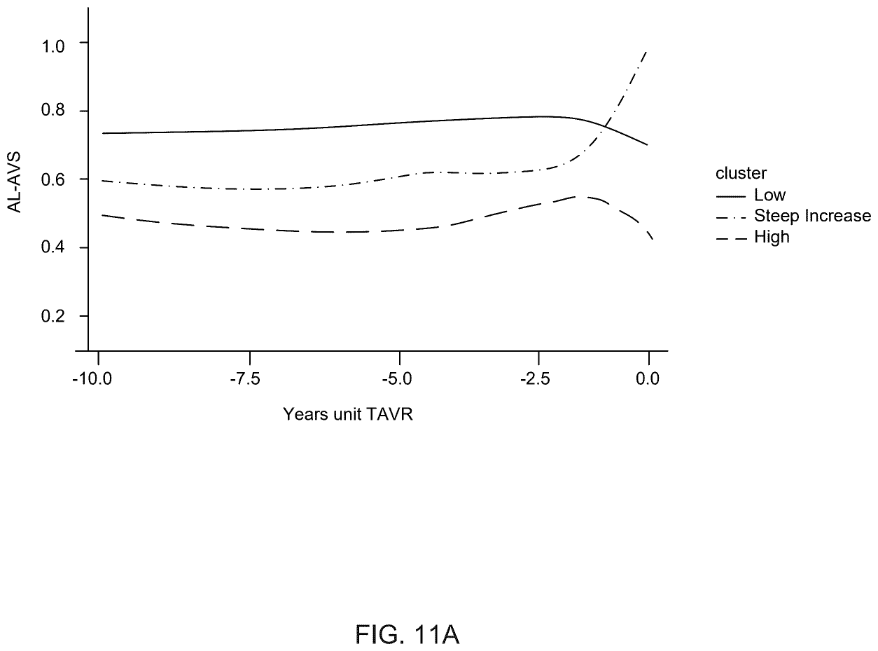

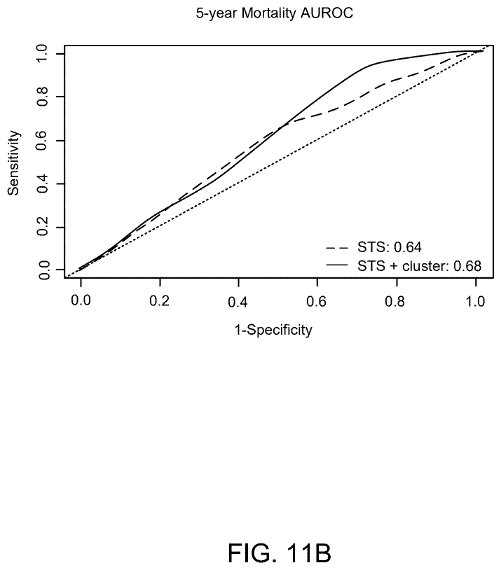

Methods: The AK-AVS model was applied to 7,860 ECGs from 2,040 patients, with scores grouped into three trajectory clusters: Low, Steep Increase, and High. See FIG. 11A. Associations between trajectory clusters and 1-year mortality, 5-year mortality (FIG. 11B), pacemaker implantation, and time to TAVR were analyzed using Cox regression models adjusted for clinical and procedural variables.

Certain adjustments to the model were evaluated to determine their predictive impact on the risk of Mortality. Briefly, the foundational system for screening an electrocardiogram (ECG) for a pattern indicative of aortic valve stenosis of the disclosure was devised to have an input module configured to receive a plurality of parameters from one or more ECG leads and an analysis module including a computer program model trained to detect a pattern predictive of aortic valve stenosis based from at least two inputs: the age of a subject associated with the ECG and the QT interval. Additional inputs have been considered and their effect on specificity and sensitivity have been considered as well. Historical ECGs were applied to the model. The trend of the model results over time indicate risk for worse outcomes post intervention. For instance, if the model scores remained high, or suddenly spiked from low to high, the patient was more likely to experience worse mortality.

TABLE 13

| | | | Model 2: Model 1 + | | | |
|---|---|---|---|---|---|---|
| | Model 1: | | STS, Agatston Score, | | Model 3: | |
| | Unadjusted | | Valve Size, Device | | Model 2 + BMI, PPM | |
| | HR | | HR | | HR | |
| | (95% CI) | P-value | (95% CI) | P-value | (95% CI) | P-value |
| Steep | 1.28 (0.95, | 0.11 | 1.38 | 0.035 | 1.4 (1.03, | 0.033 |
| Increase | 1.72) | | (1.02, 1.88) | | 1.91) | |
| (n = 482) | | | | | | |
| High | 1.43 | 0.007 | 1.52 (1.17, | 0.002 | 1.48 (1.13, | 0.005 |
| (n = 1165) | (1.1, 1.85) | | 1.98) | | 1.94) | |

### Results:

Steep Increase and High clusters had significantly higher 1-year mortality than Low (adjusted HR 1.4 and 1.48, respectively). AK-AVS could identify moderate-to-severe AS approximately 2 years before typical intervention. False-positive AK-AVS results were associated with a significantly higher risk of developing moderate/severe AS (HR 5.21, 95% CI 2.83-9.61) and heart failure (aHR 1.55, 95% CI 1.11-2.16) within 5 years (FIG. 11B).

While this invention is satisfied by configurations in many different forms, as described in detail in connection with preferred configurations of the invention, it is understood that the present disclosure is to be considered as exemplary of the principles of the invention and is not intended to limit the invention to the specific configurations illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

What is claimed is:

1. A system for screening an electrocardiogram (ECG) for a pattern indicative of aortic valve stenosis, comprising:
an input module configured to receive a plurality of parameters from one or more ECG leads;
an analysis module including a computer program model trained to detect a pattern predictive of aortic valve stenosis based from at least two inputs: the age of a subject associated with the ECG and one or more of P wave amplitude, R wave amplitude, R wave duration, S wave amplitude, S wave duration, T wave amplitude, and QT interval, and outputting a value that is predictive of aortic valve stenosis based on the pattern that is predictive of aortic valve stenosis.
an output module configured to provide an output indicative of aortic valve stenosis based on the identified pattern from the at least two inputs.

2. The system of claim 1, wherein the analysis module is configured to identify the pattern predictive of aortic valve stenosis using no more than two inputs: the age of the subject associated with the ECG and the QT interval.

3. The system of claim 1, wherein the negative predictive value of the identified pattern predictive of aortic valve stenosis exceeds 90%.

4. The system of claim 1, wherein the sensitivity of the identification of the pattern predictive of aortic valve stenosis exceeds 70%.

5. The system of claim 1, wherein the specificity of the identification of the pattern predictive of aortic valve stenosis exceeds 70%.

6. The system of claim 1, wherein the analysis module is further configured to identify a pattern predictive of aortic valve stenosis based on at least three inputs selected from the group consisting of: the age of the subject associated with the ECG, the QT interval, T amplitude from lead V4, T amplitude from lead AVL, and R amplitude from lead II.

7. The system of claim 6, wherein the pattern predictive of aortic valve stenosis comprises a reduced QT interval as a function of the age of the subject associated with the ECG.

8. The system of claim 6, wherein the pattern predictive of aortic valve stenosis comprises an increased T amplitude from lead V4 as a function of the age of the subject associated with the ECG.

9. The system of claim 1, wherein the aortic valve stenosis comprises one or more of the following: rheumatic disorder of both mitral and aortic valves, nonrheumatic aortic valve stenosis (AVS), nonrheumatic AVS with insufficiency, congenital stenosis of AVS, bicuspid aortic valve, congenital insufficiency of the aortic valve, aortic insufficiency/stenosis, nonrheumatic aortic valve disorder, unspecified other nonrheumatic AVS, moderate aortic stenosis, congenital aortic stenosis, aortic regurgitation, rheumatic aortic stenosis with insufficiency, congenital subaortic stenosis, supravalvular aortic stenosis, rheumatic aortic stenosis with insufficiency, rheumatic aortic stenosis, aortic valve disease, aortic regurgitation, and rheumatic aortic stenosis.

10. The system of claim 1, wherein the computer program model is trained on a dataset comprising at least 1,000 ECGs from subjects with at least one form of aortic valve stenosis and at least 1,000 control subjects without a diagnosis of heart disease.

11. The system of claim 1, wherein the computer program model is a feedforward neural network.

12. The system of claim 1, wherein the computer program model is trained on 62 parameters, including the age of the subject associated with the ECG, P wave amplitude, R wave amplitude, R wave duration, S wave amplitude, S wave duration, T wave amplitude, and average QT interval.

13. The system of claim 1, wherein a user directs the training of the model by dynamically adjusting the learning rate in response to plateauing of a monitored metric.

14. The system of claim 1, wherein a user directs the training of the model to prevent overfitting by monitoring validation metrics.

15. The system of claim 1, wherein the subject is at least 18 years of age.

16. The system of claim 1, wherein the subject does not have a pacemaker.

17. The system of claim 1, wherein the output module is further configured to provide a risk score indicating the probability of developing moderate or severe aortic stenosis within a predefined time period following a negative echocardiogram.

18. The system of claim 1, wherein the output module is further configured to provide a risk score indicating the probability of developing heart failure within a predefined time period following a negative echocardiogram.

19. The system of claim 1, wherein the analysis module is further configured to analyze longitudinal ECG data from a subject and to classify the subject into a risk trajectory cluster, wherein the risk trajectory cluster is associated with a distinct prognosis for mortality or adverse cardiovascular outcomes.

20. The system of claim 1, wherein the analysis module is further configured to analyze periprocedural changes in the risk score before and after aortic valve intervention, and the output module is configured to provide a prognostic indicator of one-year mortality, risk of permanent pacemaker implantation, or length of hospital stay.

21. The system of claim 1, wherein the output module is configured such that a positive risk score in the absence of echocardiographic evidence of aortic stenosis is associated with a statistically significant increased risk of developing moderate or severe aortic stenosis or heart failure within five years.

22. The system of claim 1, wherein the output module is further configured to trigger automated alerts or referrals for further diagnostic evaluation or early intervention in a hospital electronic health record system based on the output.

23. The system of claim 1, wherein the computer program model is trained and validated on datasets comprising at least 100,000 patients and is configured to maintain predictive accuracy across diverse demographic groups.

24. The system of claim 1, wherein the output module is further configured to provide a recommendation for timing of aortic valve intervention based on the subject's risk trajectory cluster and predicted clinical outcomes.

25. The system of claim 1, wherein the analysis module is further configured to provide a risk score for adverse outcomes following transcatheter aortic valve replacement, including mortality, need for permanent pacemaker, and length of hospital stay.

26. The system of claim 1, wherein the analysis module is further configured to provide a risk score for future onset of aortic stenosis in subjects with false-positive screening results, and wherein the risk score is used to guide longitudinal surveillance.
